SEQUENCE LISTING (22 sequences)

SEQ ID NO: 1 - protein, 107 aa, Artificial Sequence
  Synthetic amino acid sequence of the mature region of mouse VE1 light chain
DIQMTQTTSSLSASLGDRVTISCRTSQDIGNSLNWYQQKPDGTVKVLIYYTSRLQSGVPSRFSGSGSGTDYSLTISNLDQEDIATYFCQKGNTPPYTFGGGTKLEIK

SEQ ID NO: 2 - protein, 107 aa, Artificial Sequence
  Synthetic amino acid sequences of the mature variable regions of the HuVE1-L1  light chain
DIQMTQSPSSLSASVGDRVTITCRTSQDIGNSLNWYQQKPGKVPKLLIYYTSRLQSGVPSRFSGSGSGTDFTLTISSLQPEDVATYYCQKGNTPPYTFGQGTKLEIK

SEQ ID NO: 3 - protein, 107 aa, Artificial Sequence
  Synthetic amino acid sequence of the mature variable region of the HuVE1-L2 light chain
DIQMTQSPSSLSASVGDRVTITCRTSQDIGNSLNWYQQKPGKVPKVLIYYTSRLQSGVPSRFSGSGSGTDYTLTISSLQPEDVATYYCQKGNTPPYTFGQGTKLEIK

SEQ ID NO: 4 - protein, 107 aa, Artificial Sequence
  Synthetic amino acid sequence of the mature human acceptor V region AAS01771
DIQMTQSPSSLSASVGDRVTITCRASQGISNYLAWYQQKPGKVPKLLIYAASTLQSGVPSRFSGSGSGTDFTLTISSLQPEDVATYYCQKYNSAPYTFGQGTKLEIK

SEQ ID NO: 5 - protein, 120 aa, Artificial Sequence
  Synthetic amino acid sequence of the mature region of mouse VE1 heavy chain
QIQLVQSGPELKKTGETVKISCKASGYTFTHYGMNWVKQAPGKSLKWMGWINTYTGEPTYADDFKGRFAFSLETSASTAYLQINNLKNEDMATYFCARFGSNYEWYFDVWGAGTTVTVSS

SEQ ID NO: 6 - protein, 120 aa, Artificial Sequence
  Synthetic amino acid sequences of the mature variable regions of the HuVE1-H1 heavy chain
QVQLVQSGAEVKKPGASVKVSCKASGYTFTHYGMNWVRQAPGQGLKWMGWINTYTGEPTYADDFKGRVTFTLDTSISTAYMELSRLRSDDTAVYYCARFGSNYEWYFDVWGQGTMVTVSS

SEQ ID NO: 7 - protein, 120 aa, Artificial Sequence
  Synthetic amino acid sequence of the mature variable region of the HuVE1-H2 heavy chain
QIQLVQSGAEVKKPGASVKVSCKASGYTFTHYGMNWVRQAPGQGLKWMGWINTYTGEPTYADDFKGRFTFTLDTSISTAYMELSRLRSDDTAVYYCARFGSNYEWYFDVWGQGTMVTVSS

SEQ ID NO: 8 - protein, 122 aa, Artificial Sequence
  Synthetic amino acid sequence of the mature human acceptor V region AAC18292
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGARFGEAYDAFDIWGQGTMVTVSS

SEQ ID NO: 9 - protein, 107 aa, Artificial Sequence
  Synthetic amino acid sequence of the (mature) light chain variable region of the A2B mAb
DIQMTQTTSSLSASLGDRVTITCRASQDISNYLSWYQQKPDGTVKLLIYYTSRLHSGVPSRFSGSGSGTDYSLTISNLDQEDIATYFCQQGDTLPPTFGAGTKLELK

SEQ ID NO: 10 - protein, 118 aa, Artificial Sequence
  Synthetic amino acid sequence of the heavy chain variable region of the A2B mAb
EAQLQQSGAELVKPGASVKLSCTASGLNIKDAYIHWVKQRPEQGLEWIGRIDPANGNTKYDPKFQGKATITADTSSNTAYLQLSSLTSEDAAVYFCTYGYDGYHFDIWGQGTTLTVSS

SEQ ID NO: 11 - protein, 111 aa, Artificial Sequence
  Synthetic amino acid sequence of the mature region of mouse A2T light chain
DIVLTQSPASLAVSLGQRATISCRASKRVSTSGHSYMHWYQQKPGQPPKLLIFLASNLESGVPARFSGSGSGTDFTLNIHPVEEEDAATYYCQHSWELPWTFGGGTKLEIK

SEQ ID NO: 12 - protein, 111 aa, Artificial Sequence
  Synthetic amino acid sequences of the mature variable regions of the HuA2T-L1 light chain
DIQLTQSPSFLSASVGDRVTITCRASKRVSTSGHSYMHWYQQKPGKAPKLLIFLASNLESGVPSRFSGSGSGTEFTLTISSLQPEDFATYYCQHSWELPWTFGQGTKVEIK

SEQ ID NO: 13 - protein, 111 aa, Artificial Sequence
  Synthetic amino acid sequence of the mature variable region of the HuA2T-L2  light chain
DIQLTQSPSFLSASVGDRVTITCRASKRVSTSGHSYMHWYQQKPGKPPKLLIFLASNLESGVPSRFSGSGSGTEFTLTISSLQPEDFATYYCQHSWELPWTFGQGTKVEIK

SEQ ID NO: 14 - protein, 107 aa, Artificial Sequence
  Synthetic amino acid sequence of the mature human acceptor V region AIT39024
DIQLTQSPSFLSASVGDRVTITCRASQGISSYLAWYQQKPGKAPKLLIYTASTLQSGVPSRFSGSGSGTEFTLTISSLQPEDFATYYCQQLNSYPYTFGQGTKVEIK

SEQ ID NO: 15 - protein, 119 aa, Artificial Sequence
  Synthetic amino acid sequence of the mature region of mouse A2T heavy chain
EVKLLESGGGLVQPGGSLKLSCAASGFDFSRHWMSWLRQAPGKGLEWLGEINPESTTINYTPSLKGNFFISRDNAKNTLYLLMNKVGSEDTALYYCARPGDDFIYFDSWGQGTTLTVSS

SEQ ID NO: 16 - protein, 119 aa, Artificial Sequence
  Synthetic amino acid sequences of the mature variable regions of the HuA2T-H1 heavy chain
EVQLVESGGGLVQPGGSLRLSCAASGFDFSRHWMSWVRQAPGKGLEWLGEINPESTTINYAPSLKGRFTISRDNAKNSLYLQMNSLRAEDTAVYYCARPGDDFIYFDSWGQGTLVTVSS

SEQ ID NO: 17 - protein, 119 aa, Artificial Sequence
  Synthetic amino acid sequence of the mature variable region of the HuA2T-H2   heavy chain
EVQLVESGGGLVQPGGSLRLSCAASGFDFSRHWMSWLRQAPGKGLEWLGEINPESTTINYAPSLKGNFTISRDNAKNSLYLQMNSLRAEDTAVYYCARPGDDFIYFDSWGQGTLVTVSS

SEQ ID NO: 18 - protein, 121 aa, Artificial Sequence
  Synthetic amino acid sequence of the mature human acceptor V region AIT38751
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARVGRRLGELSADYWGQGTLVTVSS

SEQ ID NO: 19 - protein, 13 aa, Artificial Sequence
  Synthetic peptide linker
ASTKGPSVFPLAP

SEQ ID NO: 20 - protein, 13 aa, Artificial Sequence
  Synthetic peptide linker
RTVAAPSVIFIPP

SEQ ID NO: 21 - protein, 17 aa, Artificial Sequence
  Synthetic peptide linker
GGGGSGGGGSGGGGSGS

SEQ ID NO: 22 - protein, 8 aa, Artificial Sequence
  Synthetic amino acid FLAG tag
DYKDDDDK

(19) United States
(12) Patent Application Publication
(10) Pub. No.: US 2019/0352386 A1
(43) Pub. Date: Nov. 21, 2019

(54) HIGHLY POTENT MONOCLONAL ANTIBODIES TO ANGIOGENIC FACTORS

(71) Applicant: Galaxy Biotech, LLC, Sunnyvale, CA (US)

(72) Inventors: Kyung Jin Kim, Cupertino, CA (US); Hangil Park, San Francisco, CA (US); Yi Ding, Milpitas, CA (US); April Zhang, San Jose, CA (US); Lihong Wang, Hayward, CA (US); Maximiliano Vasquez, Palo Alto, CA (US)

(21) Appl. No.: 15/759,471

(22) PCT Filed: Sep. 13, 2016
(86) PCT No.: PCT/US16/51486

§ 371 Date: Mar. 12, 2018

Related U.S. Application Data
(60) Provisional application No. 62/218,226, filed on Sep. 14, 2015.

(51) Int. Cl. C07K 16/22; A61P 35/00

(52) U.S. Cl. CPC C07K 16/22; A61P 35/00; C07K 2317/76; C07K 2317/92; C07K 2317/24; C07K 2317/31; C07K 2317/732; C07K 2317/565

(57) ABSTRACT

The present invention is directed toward neutralizing monoclonal antibodies to Vascular Endothelial Growth Factor (VEGF) and angiopoietin 2 (Ang-2), pharmaceutical compositions comprising same, and methods of treatment comprising administering such a pharmaceutical composition to a patient.

### CROSS-REFERENCE TO RELATED APPLICATIONS

This application is a 371 National Stage entry of PCT Patent Application No. PCT/US2016/051486, filed Sep. 13, 2016, which claims priority to U.S. provisional application No. 62/218,226, filed Sep. 14, 2015, the entire content of which is incorporated by reference herein.

### REFERENCE TO A “SEQUENCE LISTING” SUBMITTED AS ASCII TEXT FILES VIA EFS-WEB

The Sequence Listing written in file 089367-1078640_SequenceListing.txt created on Jul. 12, 2019, 22,152 bytes, machine format IBM-PC, MS-Windows operating system, in accordance with 37 C.F.R. §§ 1.821- to 1.825, is hereby incorporated by reference in its entirety for all purposes.

### FIELD OF THE INVENTION

The present invention relates generally to the combination of monoclonal antibody (mAb) and recombinant DNA technologies for developing novel biologics, and more particularly, for example, to the production of monoclonal antibodies that bind to and neutralize Vascular Endothelial Growth Factor or Angiopoietin-2.

### BACKGROUND OF THE INVENTION

Angiogenesis is the process of new blood vessel formation from existing vasculature. Angiogenesis is required not only for normal development and tissue regeneration, but for the growth of tumors beyond 2-3 mm in size (reviewed in N. Vasudev et al., Angiogenesis 17:471-494, 2014), in order to supply the tumors with oxygen and nutrients. It was therefore proposed that inhibition of angiogenesis could suppress tumor growth (J. Folkman, N Eng J Med 285:1182-1186, 1971). Aberrant angiogenesis is also involved in other pathologic conditions including age-related macular degeneration, diabetic retinopathy and rheumatoid arthritis.

A large number of cellular factors promote angiogenesis, including vascular endothelial growth factor (VEGF), fibroblast growth factors 1 and 2 (FGF1 and FGF2), platelet derived growth factor (PDGF), placental growth factor (PGF or PIGF), insulin-like growth factor (IGF), angiopoietin 1 and 2 (Ang-1 and Ang-2), and hepatocyte growth factor (HGF) (reviewed in R. Gacche et al., Prog Biophys Mol Biol 113:333-354, 2013). The VEGF family of homologous growth factors, consisting of VEGF-A, VEGF-B, VEGF-C and VEGF-D, plays an important role by mediating endothelial cell proliferation, migration and tube formation (reviewed in T. Veikkola et al., Semin Cancer Biol 9: 211-220, 1999). Of these, VEGF-A is the best studied and plays a key role in normal and neoplastic angiogenesis; VEGF without a letter identifier shall mean VEGF-A herein.

VEGF (i.e., VEGF-A) is a homodimeric glycoprotein consisting of two identical 23 kDa monomers. There are several alternatively spliced isoforms of human VEGF, including VEGF₁₂₁, VEGF₁₆₅, VEGF₁₈₉, and VEGF₂₀₆ (N. Ferrara et al. Nature Med 9:669-676, 2003). Of these, VEGF₁₆₅ is the most abundant and mitogenic isoform and corresponds to the 23 kDa subunit. VEGF₁₈₉ and VEGF₂₀₆ bind to heparin and therefore to the extracellular matrix; VEGF₁₆₅ is diffusable (structure and biology of VEGF reviewed in Q. T. Ho et al., Int J Biochem Cell Biol 39:1349-1357, 2007). The VEGF family members bind to three tyrosine kinase cellular receptors: VEGFR1 (Flt-1), VEGFR2 (Flk-1; KDR) and VEGFR3, with VEGF-A primarily signalling through VEGFR2 (reviewed in C. Fontanella et al., Ann Transl Med 2:123, 2014), so VEGFR2 will also be called VEGFR herein. Binding of VEGF to VEGFR2 leads to receptor dimerization, autophosphorylation, and activation of the MEK-MAP and PI3K-AKT signalling pathways, causing cellular proliferation and endothelial cell survival.

Because VEGF is a key driver of angiogenesis in tumors, inhibitors of VEGF have the potential to treat cancer. A monoclonal antibody (mAb) to human VEGF was effective at inhibiting the growth of human tumor xenografts in mice (K. J. Kim et al., Nature 362:841-844, 1993). A humanized form of this antibody, bevacizumab (Avastin®), was shown in a series of clinical trials to improve patient survival for several types of cancer (reviewed in N. Vasudev, op. cit.) and has been approved for treatment of types of colorectal, lung, renal, cervical, and ovarian cancer in combination with various other drugs, and for glioblastoma (Avastin® package label). However, the progression-free or overall survival benefits of bevacizumab are generally quite small, usually a few months (R. S. Kerbel, The Breast S3: S56-S60, 2011). In an attempt to improve upon bevacizumab, other anti-VEGF mAbs have been generated including MAb7392 (WO 2011/159704), the humanized rabbit mAb hEBV321 (Y. Yu et al., PLOS ONE 5:e9072, 2010; U.S. Pat. No. 7,803,371; US 2012/0231011), the humanized mAb Y0317 (Y. Chen et al., J Mol Biol: 293:865-81, 1999), and the human mAbs B20.4.1 and B20.4.1.1 (US 2009/0142343). However these mAbs have not been approved for marketing.

The angiopoietin family of cytokines consists of Angiopoietin 1 (Ang-1), Angiopoietin 2 (Ang-2) and in humans the less studied Angiopoietin 4 (for reviews of the structure and function of angiopoietins and their receptors, see M. Thomas et al. Angiogenesis 12:125-137, 2009 and E. Fagiani et al., Cancer Letters 328: 18-26, 2013). The angiopoietins are secreted glycoproteins with a dimeric molecular weight of 70-75 kDa, but also form heterogenous multimers such as trimers and tetramers; such oligomerization is necessary for receptor activation. The angiopoietins bind to and signal through the Tie-2 tyrosine kinase receptor; Tie-1 is an orphan receptor that is able to heterodimerize with Tie-2 and modulate signal transduction. Whereas Ang-1 signals positively through Tie-2, Ang-2 has been reported as an agonist or antagonist depending on context. The angiopoietins act on the vasculature in a complex manner. Whereas Ang-1 generally stabilizes blood vessels and is critical for blood vessel development in the embryo, Ang-2 released by endothelial cells can act as a competitive antagonist to Ang-1 and thus promote disassociation of pericytes from endothial cells, sprouting of tip cells and, in the presence of VEGF, angiogenesis.

Several human mAbs that specifically bind and neutralize Ang-2 have been generated using phage display or transgenic mice, including Ab536 (J. Oliner et al., Cancer Cell 6:507-16, 2004), MEDI-3617 (C. C. Leow et al., Int J Oncol 40:1321-30, 2012, and A. Buchanan et al., MAbs 5:255-62, 2013), LCO6 (M. Thomas et al., PLoS One. 8:e54923, 2013) and REGN910 (C. Daly et al., Cancer Res 73:108-18, 2012). These mAbs block binding of Ang-2 to Tie-2, inhibit angiogenesis, and inhibit tumor xenograft growth in various models. A bispecific antibody binding to both VEGF and Ang-2 has also been reported (Y. Kienast, Clin Cancer Res 19:6730-6740, 2013).

### SUMMARY OF THE CLAIMED INVENTION

In one embodiment, the invention provides a neutralizing monoclonal antibody (mAb) to human Vascular Endothelial Growth Factor (VEGF) that has the same epitope as the VE1 antibody disclosed herein. Exemplary antibodies are VE1 and mAbs that comprise a light chain variable region having three CDRs from the light chain variable region sequence of VE1 and a heavy chain variable region having three CDRs from the heavy chain variable region sequence of VE1, for example chimeric and humanized forms of VE1, such as mAbs comprising the humanized light and heavy chains listed in FIG. 3. The mAb inhibits at least one and preferably several or all biological activities of VEGF including binding to its cellular receptor. Advantageously, the anti-VEGF mAb inhibits growth of a human tumor xenograft in a mouse. A pharmaceutical composition comprising any such mAb is also provided, as well as a method of treating a patient having a disease, e.g., cancer, by administering such a pharmaceutical composition.

In another embodiment, the invention provides a neutralizing monoclonal antibody (mAb) to human Angiopoietin 2 (Ang-2) that has the same epitope as the A2T antibody disclosed herein. Exemplary antibodies are A2T and mAbs that comprise a light chain variable region having three CDRs from the light chain variable region sequence of A2T and a heavy chain variable region having three CDRs from the heavy chain variable region sequence of A2T, for example chimeric and humanized forms of A2T, such as mAbs comprising the humanized light and heavy chains listed in FIG. 13. The mAb inhibits at least one and preferably several or all biological activities of Ang-2 including binding to its cellular receptor and stimulation of angiogenesis. A pharmaceutical composition comprising any such mAb is also provided, as well as a method of treating a patient having a disease, e.g., cancer, by administering such a pharmaceutical composition.

Bispecific antibodies that incorporate one or more binding domains from any of the above-mentioned antibodies, together with one or more binding domains from a different antibody with another target, are also provided. In preferred embodiments, the other target is human Hepatocyte Growth Factor (HGF), and the different antibody may be HuL2G7, or the other target is human FGF2 and the different antibody may be a humanized GAL-F2 mAb. In exemplary embodiments, one or more binding domains are from a humanized VE1 mAb and one or more binding domains are from a humanized or human mAb to Ang-2, for example a humanized A2T mAb. Often, the bispecific antibody is a homodimer of monomers, each of which comprises a first binding domain that binds to VEGF and a second binding domain that binds to HGF or FGF2 or Ang-2.

### BRIEF DESCRIPTION OF THE DRAWINGS

FIG. 1. Schematic diagrams of the Bs(scFv)₄-IgG bispecific antibody format. The upper diagram (A) shows individual variable and constant regions; the lower diagram (B) shows domains formed by folding together of each light chain region with the respective heavy chain region. V_{H}1 (respectively V_{L}1)=heavy (resp. light) chain variable region of first antibody; and similarly for V_{H}2 and VII of second antibody. C_{H}1, C_{H}2, C_{H}3 (resp. C_{L})=heavy (resp. light) chain constant region domains; V1 (resp. V2)=full variable domain of first (resp. second) antibody.

FIG. 2. (A) ELISA assay showing that VE1 but not control mouse mAb mIgG captures VEGF. (B) ELISA assay showing that VE1 blocks binding of VEGF to VEGFR better than A4.6.1.

FIG. 3. Amino acid sequences of the mature variable regions of the HuVE1-L1 and HuVE1-L2 light chains (A) and HuVE1-H1 and HuVE1-H2 heavy chains (B) are shown aligned with mouse VE1 and human acceptor V regions. The CDRs are underlined in the VE1 sequences, and the amino acids substituted with mouse VE1 amino acids are double underlined in the HuVE1 sequences. The 1-letter amino acid code and Kabat numbering system are used for both the light and heavy chain herein.

FIG. 4. ELISA assays comparing the binding (A) and receptor blocking (B) activities of ChVE1 and HuVE1 variants #1, #2, #3, #4, and negative control antibody hIgG.

FIG. 5. ELISA assays comparing the binding (A) and receptor blocking (B) activities of HuVE1 variants #3 and #4 with bevacizumab and negative control hIgG.

FIG. 6. (A) Biological assay showing that HuVE1 #4 inhibits VEGF-induced proliferation of human umbilical vascular endothelial cells (HUVEC) better than bevacizumab does. (B) ELISA assay comparing the ability of the indicated anti-VEGF mAbs to block binding of VEGF to VEGFR2.

FIG. 7. (A) ELISA assays showing that HuVE1 #4 (and bevacizumab) (A) bind to VEGF-A (VEGF) but not to VEGF-B, VEGF-C, VEGF-D, HGF, and FGF2, and (B) bind to the VEGF-165, VEGF-121 and VEGF-189 isoforms of VEGF.

FIG. 8. (A) Schematic diagram of human (Hu or h)/mouse (Mu or m) chimeric forms of VEGF. Shaded, human sequence; hatched, mouse sequence; KF, kappa-flag. (B) ELISA assay of binding of HuVE1 #4 and bevacizumab to each of the constructs in (A).

FIG. 9A,B. Binding of HuVE1 #4 and bevacizumab to various mutants of VEGF as labeled. WT; wild-type VEGF.

FIG. 10. (A) ELISA assay of binding of the indicated anti-Ang-2 mAbs to human (h), mouse (m) and cynomolgus monkey (cyno) Ang-2 constructs. (B) ELISA assay of binding of the indicated anti-Ang-2 mAbs to human, mouse, human-mouse chimeric (h/m) and mouse-human (m/h) chimeric Ang-2 constructs.

FIG. 11. ELISA assay comparing the ability of the indicated mAbs to block binding of (human) Ang-2 to (human) Tie-2.

FIG. 12. Amino acid sequences of the (mature) light (A) and heavy (B) chain variable regions of the A2B mAb.

FIG. 13. Amino acid sequences of the mature variable regions of the HuA2T-L1 and HuA2T-L2 light chains (A) and HuA2T-H1 and HuA2T-H2 heavy chains (B) are shown aligned with mouse A2T and human acceptor V regions. The CDRs are underlined in the A2T sequences, and the amino acids substituted with mouse A2T amino acids are double underlined in the HuA2T sequences. The amino acids at position 60 converted from the mouse T to the human A to eliminate a potential glycosylation site are shown shaded.

FIG. 14. ELISA assays comparing the ability of the indicated HuA2T variants to bind to Ang-2 (A) and inhibit binding of Ang-2 to Tie-2 (B).

FIG. 15. ELISA assays comparing the ability of the indicated HuA2T variants to bind to Ang-2 (A) and inhibit binding of Ang-2 to Tie-2 (B).

FIG. 16. (A) ELISA assay comparing the ability of the indicated anti-Ang-2 mAbs to inhibit binding of Ang-2 to Tie-2. (B) Assay comparing the ability of the indicated anti-Ang-2 mAbs to inhibit Ang-2 induced phosphorylation of Tie-2 in HEK293-Tie-2 cells.

FIG. 17. (A) ELISA assay showing the ability of the B-HuA2T/HuVE1 bispecific antibody but not HuVE1 to simultaneously bind Ang-2 and VEGF. (B) ELISA assay comparing the ability of B-HuA2T/HuVE1, HuVE1 and bevacizumab to bind VEGF.

FIG. 18. ELISA assays comparing the ability of B-HuA2T/HuVE1 and HuVE1 to inhibit binding of VEGF to VEGFR2 (A), and of B-HuA2T/HuVE1 and HuA2T to inhibit binding of Ang-2 to Tie-2 (B).

FIG. 19. (A) Growth of COLO 205 xenografts in mice treated with VE1 (5 mg/kg) or vehicle (PBS) alone, twice per week. (B) Growth of COLO 205 xenografts in mice treated with HuVE1 #3 (5 mg/kg) or PBS alone, twice per week.

FIG. 20. (A) Growth of primary liver tumor xenografts in mice treated with HuVE1 or bevacizumab (2.5 mg/kg) or vehicle (PBS) alone, twice per week. (B) Growth of RPMI 4788 colon tumor xenografts in mice treated with HuVE1 or bevacizumab (1 mg/kg) or PBS alone, on days 6 and 9 as indicated by arrows.

FIG. 21. Growth of primary breast tumor xenografts in mice treated with HuVE1 or bevacizumab (5 mg/kg) or vehicle (PBS) alone, once per week.

### DETAILED DESCRIPTION OF THE PREFERRED EMBODIMENTS

### 1. Antibodies

As used herein, “antibody” means a protein containing one or more domains capable of binding an antigen, where such domain(s) are derived from or homologous to the variable domain of a natural antibody. A monoclonal antibody (“mAb”) is simply a unique species of antibody, in contrast to a mixture of different antibodies. The antibodies described herein are generally monoclonal, unless otherwise indicated by the context. An “antigen” of an antibody means a compound to which the antibody specifically binds and is typically a polypeptide, but may also be a small peptide or small-molecule hapten or carbohydrate or other moiety. Examples of antibodies include natural, full-length tetrameric antibodies; antibody fragments such as Fv, Fab, Fab′ and (Fab′)2; single-chain (scFv) antibodies (Huston et al., Proc Natl Acad Sci USA 85:5879, 1988; Bird et al., Science 242:423, 1988); single-arm antibodies (Nguyen et al., Cancer Gene Ther 10:840, 2003); and bispecific, chimeric and humanized antibodies, as these terms are further explained below. Antibodies may be derived from any vertebrate species, including chickens, rodents (e.g., mice, rats and hamsters), rabbits, primates and humans. An antibody comprising a constant domain may be of any of the known isotypes IgG, IgA, IgM, IgD and IgE and their subtypes, i.e., human IgG1, IgG2, IgG3, IgG4 and mouse IgG1, IgG2a, IgG2b, and IgG3, and their allotypes and isoallotypes, including combinations of residues occupying polymorphic positions in allotypes and isoallotypes. An antibody can also be of chimeric isotype, that is, one or more of its constant (C) regions can contain regions from different isotypes, e.g., a gamma-1 C_{H}1 region together with hinge, C_{H}2 and/or C_{H}3 domains from the gamma-2, gamma-3 and/or gamma-4 genes. The antibody may also contain replacements in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc Natl Acad Sci USA 103:4005, 2006), or to prolong half-life in humans (see, e.g., Hinton et al., J Biol Chem 279:6213, 2004).

A natural antibody molecule is generally a tetramer consisting of two identical heterodimers, each of which comprises one light chain paired with one heavy chain. Each light chain and heavy chain consists of a variable (V_{L} or V_{H}, or simply V) region followed by a constant (CL or C_{H}, or simply C) region. The C_{H} region itself comprises C_{H}1, hinge (H), C_{H}2, and C_{H}3 regions. In 3-dimensional (3D) space, the V_{L} and V_{H} regions fold up together to form a V domain, which is also known as a binding domain since it binds to the antigen. The CL region folds up together with the C_{H}1 region, so that the light chain V_{L}-CL and the V_{H}-C_{H}1 region of the heavy chain together form a part of the antibody known as a Fab: a naturally “Y-shaped” antibody thus contains two Fabs, one from each heterodimer, forming the arms of the Y. The C_{H}2 region of one heterodimer is positioned opposite the C_{H}2 region of the other heterodimer, and the respective C_{H}3 regions fold up with each other, forming together the single Fc domain of the antibody (the base of the Y), which interacts with other components of the immune system.

Within each light or heavy chain variable region, there are three short segments (averaging about 10 amino acids in length) called the complementarity determining regions (“CDRs”). The six CDRs in an antibody variable domain (three from the light chain and three from the heavy chain) fold up together in 3D space to form the actual antibody binding site which locks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework, which forms the environment for the CDRs. Chothia et al., J Mol Biol 196:901, 1987, have defined the related concept of hypervariable regions or loops determined by structure.

As used herein, a “genetically engineered” mAb is one for which the genes have been constructed or put in an unnatural environment (e.g., human genes in a mouse or on a bacteriophage) with the help of recombinant DNA techniques, and therefore includes chimeric antibodies and humanized antibodies, as described below, but would not encompass a mouse or other rodent mAb made with conventional hybridoma technology. A chimeric antibody (or respectively chimeric antibody light or heavy chain) is an antibody (or respectively antibody light or heavy chain) in which the variable region of a mouse (or other non-human species) antibody (or respectively antibody light or heavy chain) is combined with the constant region of a human antibody; their construction by means of genetic engineering is well-known. Such antibodies retain the binding specificity of the mouse antibody, while being about two-thirds human.

A humanized antibody is a genetically engineered antibody in which CDRs from a non-human “donor” antibody (e.g., chicken, mouse, rat, rabbit or hamster) are grafted into human “acceptor” antibody sequences, so that the humanized antibody retains the binding specificity of the donor antibody (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. Nos. 5,859,205 6,881,557; Foote, U.S. Pat. No. 6,881,557). The acceptor antibody sequences can be, for example, a mature human antibody sequence, a consensus sequence of human antibody sequences, a germline human antibody sequence, or a composite of two or more such sequences. Thus, a humanized antibody is an antibody having some or all CDRs entirely or substantially from a donor antibody and variable region framework sequences and constant regions, if present, entirely or substantially from human antibody sequences. Similarly, a humanized light chain (respectively heavy chain) has at least one, two and usually all three CDRs entirely or substantially from a donor antibody light (resp. heavy) chain, and a light (resp. heavy) chain variable region framework and light (resp. heavy) chain constant region, if present, substantially from a human light (resp. heavy) acceptor chain. A humanized antibody generally comprises a humanized heavy chain and a humanized light chain. A CDR in a humanized antibody is substantially from a corresponding CDR in a non-human antibody when at least 85%, 90%, 95% or 100% of corresponding amino acids (as defined by Kabat) are identical between the respective CDRs. The variable region framework or constant region of an antibody chain are substantially from a human variable region or human constant region respectively when at least 85%, 90%, 95% or 100% of corresponding amino acids (as defined by Kabat) are identical.

Here, as elsewhere in this application, percentage sequence identities are determined with antibody sequences maximally aligned by the Kabat numbering convention (Eu index for the C_{H} region). After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

In order to retain high binding affinity in a humanized antibody, at least one of two additional structural elements can be employed. See, U.S. Pat. Nos. 5,530,101 and 5,585,089, incorporated herein by reference, which provide detailed instructions for construction of humanized antibodies. In the first structural element, the framework of the heavy chain variable region of the acceptor or humanized antibody is chosen to have high sequence identity (between 65% and 95%) with the framework of the heavy chain variable region of the donor antibody, by suitably selecting the acceptor antibody heavy chain from among the many known human antibodies. In the second structural element, in constructing the humanized antibody, selected amino acids in the framework of the human acceptor antibody (outside the CDRs) are replaced with corresponding amino acids from the donor antibody, in accordance with specified rules. Specifically, the amino acids to be replaced in the framework are chosen on the basis of their ability to interact with the CDRs. For example, the replaced amino acids can be adjacent to a CDR in the donor antibody sequence or within 4-6 angstroms of a CDR in the humanized antibody as measured in 3-dimensional space.

Other approaches to design humanized antibodies may also be used to achieve the same result as the methods in U.S. Pat. Nos. 5,530,101 and 5,585,089 described above, for example, “superhumanization” (see Tan et al. J Immunol 169:1119, 2002, and U.S. Pat. No. 6,881,557) or the method of Studnicak et al., Protein Eng 7:805, 1994. Moreover, other approaches to produce genetically engineered, reduced-immunogenicity mAbs include “reshaping”, “hyperchimerization” and veneering/resurfacing, as described, e.g., in Vaswami et al., Annals of Allergy, Asthma and Immunology 81:105, 1998; Roguska et al. Protein Eng 9:895, 1996; and U.S. Pat. Nos. 6,072,035 and 5,639,641. Veneered antibodies are made more human-like by replacing specific amino acids in the variable region frameworks of the non-human donor antibody that may contribute to B- or T-cell epitopes, for example exposed residues (Padlan, Mol Immunol 28:489, 1991). Other types of genetically engineered antibodies include human antibodies made using phage display methods (Dower et al., WO91/17271; McCafferty et al., WO92/001047; Winter, WO92/20791; and Winter, FEBS Lett 23:92, 1998, each of which is incorporated herein by reference) or by using transgenic animals (Lonberg et al., WO93/12227; Kucherlapati WO91/10741, each of which is incorporated herein by reference).

The terms “antibody” or “mAb” also encompass bispecific antibodies. A “bispecific antibody” is an antibody that contains a first domain binding to a first antigen and a second (different) domain binding to a second antigen, where the first and second domains are derived from or homologous to variable domains of natural antibodies. The first antigen and second antigen may be the same antigen, in which case the first and second domains can bind to different epitopes on the antigen. The term bispecific antibody encompasses multispecific antibodies, which in addition to the first and second domains contain one or more other domains binding to antigens and derived from or homologous to variable domains of natural antibodies. The term bispecific antibody also encompasses an antibody containing a first binding domain derived from or homologous to a variable domain of a natural antibody, and a second binding domain derived from another type of protein, e.g., the extracellular domain of a receptor, (a “bispecific antibody-immunoadhesin”).

Bispecific antibodies have been produced in a variety of forms (see, e.g., Kontermann, MAbs 4:182-197, 2012 and references cited therein), for example single chain variable fragment (scFv), Fab-scFv, and scFv-scFv fusion proteins (Coloma et al., Nat Biotechnol 15:125-6, 1997; Lu et al., J Immunol Methods 267:213-26, 2002; Mallender, J Biol Chem 269:199-206, 1994), Bs(scFv)4-IgG (Zuo et al., Protein Eng 13: 361-367, 2000), double variable domain antibodies (Wu et al., Nat Biotechnol 25:1290-7, 2007), and diabodies (Holliger et al., Proc Natl Acad Sci USA 90:6444-8, 1993). Bispecific F(ab′)2 antibody fragments have been produced by chemical coupling (Brennan et al., Science 229:81, 1985) or by using leucine zippers (Kostelny et al., J Immunol 148:1547-53, 1992). A more naturally shaped bispecific antibody, with each heavy chain—light chain pair having a different V region, can be made, e.g., by chemically cross-linking the two heavy chain—light chain pairs produced separately (Karpovsky et al., J Exp Med 160:1686-701, 1984), Naturally shaped bispecific antibodies can also be produced by expressing both required heavy chains and light chains in a single cell, made by fusing two hybridoma cell lines (a “quadroma”; Milstein et al., Nature 305: 537-40) or by transfection. Association of the correct light and heavy chains expressed in a cell to form the desired bispecific antibody can be promoted by using “knobs-into-holes” technology (Ridgway et al., Protein Eng 9:617-21, 1996; Atwell et al., J Mol Biol 270:26-35, 1997; and U.S. Pat. No. 7,695,936); optionally with exchange or “crossing over” of heavy chain and light chain domains within the antigen binding fragment (Fab) of one light chain—heavy chain pair, thus creating bispecific antibodies called “CrossMabs” (Schaefer et al., Proc Natl Acad Sci USA 108:11187-92, 2011; WO 2009/080251; WO 2009/080252; WO 2009/080253).

An antibody is said to bind “specifically” to an antigen if it binds to a significantly greater extent than irrelevant antibodies not binding the antigen, and thus typically has binding affinity (Kₐ) of at least about 10⁶ but preferably 10⁷, 10⁸, 10⁹ or 10¹⁰ M⁻¹ for the antigen. Generally, when an antibody is said to bind to an antigen, specific binding is meant. If an antibody is said not to bind an antigen, it is meant that any signal indicative of binding is not distinguishable within experimental error from the signal of irrelevant control antibodies. The epitope of a mAb is the region of its antigen to which the mAb binds. Two antibodies are judged to bind to the same or overlapping epitopes if each competitively inhibits (blocks) binding of the other to the antigen. Competitively inhibits binding means that a 1× or 5× excess of one antibody inhibits binding of the other by at least 50% but preferably 75%, or that a 10×, 20× or 100× excess of one antibody inhibits binding of the other by at least 75% but preferably 90% or even 95% or 99% as measured in a competitive binding assay (see, e.g., Junghans et al., Cancer Res 50:1495, 1990). One mAb (the second mAb) is said to “fully” compete for binding an antigen with another mAb (the first mAb) if the inhibitory concentration 50 (IC50) of the second mAb to inhibit binding (of the first mAb) is comparable to, that is, within 2-fold or 3-fold, of the IC50 of the first mAb to inhibit binding of itself, in competitive binding assays. A second mAb is said to “partially” compete for binding an antigen with a first mAb if the IC50 of the second mAb to inhibit binding (of the first mAb) is substantially greater than, e.g., greater than 3-fold or 5-fold or 10-fold, the IC50 of the first mAb to inhibit binding. In general, two mAbs have the same epitope on an antigen if each fully competes for binding to the antigen with the other, and have overlapping epitopes if at least one mAb partially competes for binding with the other mAb. Alternatively, two antibodies have the same epitope if essentially all amino acid mutations in the antigen that reduce or eliminate binding of one antibody reduce or eliminate binding of the other, while two antibodies have overlapping epitopes if some but not all amino acid mutations that reduce or eliminate binding of one antibody reduce or eliminate binding of the other.

### 2. Anti-VEGF and Anti-Ang-2 Antibodies

When reference is made to a growth factor or receptor herein, such as VEGF, Ang-2, HGF and FGF2, the human form of the growth factor or receptor is meant, unless otherwise specified.

A monoclonal antibody that binds VEGF, i.e., an anti-VEGF mAb (or respectively a mAb that binds Ang-2, i.e., an anti-Ang-2 mAb) is said to neutralize VEGF (respectively Ang-2), or be neutralizing, if the binding partially or completely inhibits one or more biological activities of VEGF (respectively Ang-2), i.e., when the mAb is used as a single agent. Among the biological properties of VEGF that a neutralizing antibody may inhibit are the ability of VEGF to bind to its cellular receptor, to induce phosphorylation of its receptor, and to induce proliferation of human umbilical vascular endothelial cells (HUVEC) or induce angiogenesis. Among the biological properties of Ang-2 that a neutralizing antibody may inhibit are the ability of Ang-2 to bind to its cellular receptor, to induce phosphorylation of its receptor, and to induce angiogenesis. A neutralizing mAb of the invention at a concentration of, e.g., 0.01, 0.1, 0.5, 1, 2, 5, 10, 20 or 50 μg/ml inhibits a biological function of VEGF (respectively Ang-2) by about at least 50% but preferably 75%, more preferably by 90% or 95% or even 99%, and most preferably approximately 100% (essentially completely) as assayed by methods described under Examples or known in the art. Typically, the extent of inhibition is measured when the amount of VEGF (respectively Ang-2) used is just sufficient to fully stimulate the biological activity, or is 0.05, 0.1, 0.5, 1, 3 or 10 μg/ml. Preferably, the mAb neutralizes not just one but two, three or several of the biological activities listed above; for purposes herein, a mAb that used as a single agent neutralizes all the biological activities of VEGF (respectively Ang-2) is called “fully neutralizing”, and such mAbs are most preferable.

Anti-VEGF mAbs of the invention are preferably specific for VEGF (i.e., VEGF-A), that is they do not (specifically) bind, or only bind to a much lesser extent (e.g., less than ten-fold as well), proteins that are related to VEGF such as VEGF-B, VEGF-C and VEGF-D as well as other angiogenic factors, e.g., HGF and FGF2. Similarly, Anti-Ang-2 mAbs of the invention are preferably specific for Ang-2, that is they do not (specifically) bind or only bind to a much lesser extent (e.g., less than ten-fold as well), proteins that are related to Ang-2 such as Ang-1 and Ang-4 as well as other angiogenic factors such as HGF and FGF2. The mAbs of the invention typically have a binding affinity (Kₐ) for their specific target of at least 10⁷ M⁻¹ but preferably 10⁸ M⁻¹ or higher, and most preferably 10⁹ M⁻¹ or higher or even 10¹⁰ M⁻¹ or higher. The anti-VEGF mAbs bind human VEGF and the Anti-Ang-2 mAbs bind human Ang-2, but advantageously also VEGF (respectively Ang-2) from other species, e.g., mice or non-human primates such as cynomolgus monkeys, ideally with binding affinity similar to (e.g., within 10-fold) the binding affinity to human VEGF (respectively human Ang-2). MAbs of the invention include all the various forms of antibodies described above, including bispecific antibodies having a binding domain that binds VEGF or Ang-2. The sequence of human VEGF is provided in Swiss-Prot P15692, of which the first 26 residues are a signal peptide removed in mature VEGF-A.

The anti-VEGF mAb VE1 described herein is an example of the invention. Neutralizing mAbs with the same, or overlapping, epitope as VE1 provide other examples. Neutralizing anti-VEGF mAbs that are chimeric, humanized or human, e.g., a chimeric or humanized form of VE1 such as HuVE1, are especially preferred embodiments. In other preferred embodiments, the mAb is a bispecific antibody comprising one or more binding domains from an anti-VEGF mAb of the invention (e.g., VE1 or a humanized form of VE1) that has one or more of the properties mentioned above (e.g., neutralizing VEGF), and a second binding domain from a mAb that optionally binds and neutralizes HGF (e.g., the L2G7 mAb or a humanized form of it such as HuL2G7, as described in U.S. Pat. Nos. 7,220,410 and 7,632,926) or FGF2 (e.g., the GAL-F2 mAb or a humanized form of it, as disclosed in U.S. Pat. No. 8,101,725). Most preferably, the anti-VEGF mAb inhibits growth of a human tumor xenograft in a mouse as assessed by any of the assays in the Examples or otherwise known in the art. MAbs that have CDRs that individually or collectively are at least 90%, 95% or 98% or completely identical to the CDRs of VE1 in amino acid sequence and that maintain its functional properties, or which differ from VE1 by a small number of functionally inconsequential amino acid substitutions (e.g., conservative substitutions, as defined below), deletions, or insertions are also included in the invention.

The anti-Ang-2 mAbs A2T and A2B described herein are also examples of the invention. Neutralizing mAbs with the same, or overlapping, epitope as either A2T or A2B provide other examples. Neutralizing anti-Ang-2 mAbs that are chimeric, humanized or human, e.g., chimeric or humanized forms of A2T or A2B such as HuA2T, are especially preferred embodiments. In particular embodiments, the mAb is a bispecific antibody comprising one or more binding domains from an anti-Ang-2 mAb of the invention (e.g., A2T or A2B or their humanized forms) that has one or more of the properties mentioned above (e.g., neutralizing Ang-2), and a second binding domain from another mAb, such as the anti-HGF and anti-FGF2 mAbs mentioned above. Ideally, the anti-Ang-2 mAb inhibits growth of a human tumor xenograft in a mouse as assessed by any of the assays in the Examples or otherwise known in the art. MAbs that have CDRs that individually or collectively are at least 90%, 95% or 98% or completely identical to the CDRs of A2T or A2B in amino acid sequence and that maintain its functional properties, or which differ from A2T or A2B by a small number of functionally inconsequential amino acid substitutions (e.g., conservative substitutions, as defined below), deletions, or insertions are also included in the invention.

Once a single, archetypal anti-VEGF or anti-Ang-2 mAb, for example VE1 or A2T respectively, has been isolated that has the desired properties described herein, it is straightforward to generate other mAbs with similar properties by using art-known methods, including mAbs that compete with VE1 for binding to VEGF, and/or have the same epitope as VE1. For example, mice may be immunized with VEGF, hybridomas produced, and the resulting mAbs screened for the ability to compete with VE1 for binding to VEGF. Mice can also be immunized with a smaller fragment of VEGF containing the epitope to which VE1 binds. The epitope can be localized by, e.g., screening for binding to a series of overlapping peptides spanning VEGF. Mouse mAbs generated in these ways can then be humanized. Alternatively, the method of Jespers et al., Biotechnology 12:899, 1994, which is incorporated herein by reference, may be used to guide the selection of mAbs having the same epitope and therefore similar properties to VE1. Using phage display, first the heavy chain of VE1 is paired with a repertoire of (preferably human) light chains to select a VEGF-binding mAb, and then the new light chain is paired with a repertoire of (preferably human) heavy chains to select a (preferably human) VEGF-binding mAb having the same epitope as VE1. Alternatively variants of VE1 can be obtained by mutagenesis of cDNA encoding the heavy and light chains of VE1. The same procedures may be applied to develop mAbs that compete with A2T or A2B for binding to Ang-2 and/or have the same epitope as A2T or A2B.

Preferred anti-VEGF mAbs of the invention, such as HuVE1, bind to an epitope that is different from, i.e., not identical to, the epitope of bevacizumab, although the epitopes may overlap so the antibody competes with bevacizumab for binding to VEGF. Specifically, one or amino acid substitutions in VEGF that substantially impair binding of bevacizumab to VEGF may not do so, or do so to the same extent, for the current mAbs, or vice versa. Preferred antibodies of the invention have binding affinity for VEGF at least 2-fold, but more preferably 3-fold, 4-fold, 5-fold, 6-fold, 7-fold, 8-fold or even 10-fold higher than bevacizumab. Similarly, preferred antibodies of the invention inhibit binding of VEGF to VEGFR2 at least 2-fold, but more preferably 3-fold, 4-fold, 5-fold, 6-fold, 7-fold, 8-fold or even 10-fold better than bevacizumab, typically measured by the ratio of the inhibitory concentration-50% (IC50) for inhibition by bevacizumab to the IC50 for inhibition by the preferred antibody.

Genetically engineered mAbs, e.g., chimeric or humanized or bispecific mAbs, may be expressed by a variety of art-known methods. For example, genes encoding their light and heavy chain V regions may be synthesized from overlapping oligonucleotides and inserted together with available C regions into expression vectors (e.g., commercially available from Invitrogen) that provide the necessary regulatory regions, e.g., promoters, enhancers, poly A sites, etc. Use of the CMV promoter-enhancer is preferred. The expression vectors may then be transfected using various well-known methods such as lipofection or electroporation into a variety of mammalian cell lines such as CHO or non-producing myelomas including Sp2/0 and NS0, and cells expressing the antibodies selected by appropriate antibiotic selection. See, e.g., U.S. Pat. No. 5,530,101. Larger amounts of antibody may be produced by growing the cells in commercially available bioreactors.

Once expressed, the mAbs of the invention including bispecific mAbs may be purified according to standard procedures of the art such as microfiltration, ultrafiltration, protein A or G affinity chromatography, size exclusion chromatography, anion exchange chromatography, cation exchange chromatography and/or other forms of affinity chromatography based on organic dyes or the like. Substantially pure antibodies of at least about 90 or 95% homogeneity are preferred, and 98% or 99% or more homogeneity most preferred, for pharmaceutical uses. It is also understood that when the mAb is manufactured by conventional procedures, one to several amino acids at the amino or carboxy terminus of the light and/or heavy chain, such as the C-terminal lysine of the heavy chain, may be missing or derivatized in a proportion or all of the molecules, and such a composition is still considered to be the same mAb.

### 3. Bispecific Antibodies

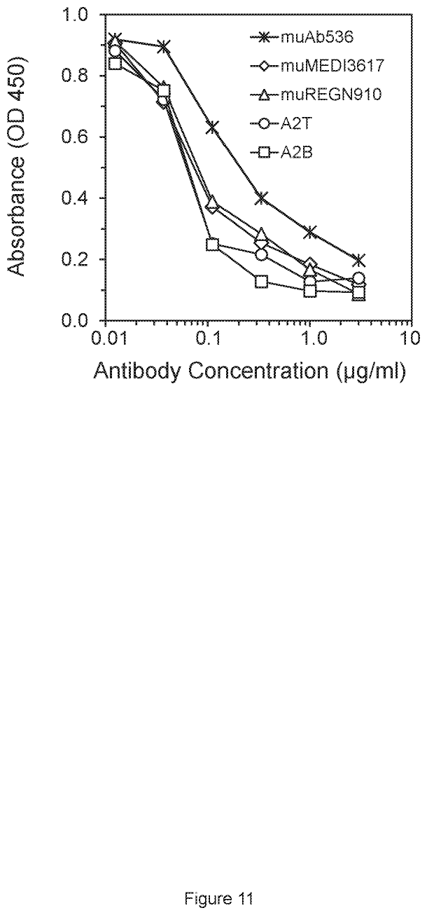

Bispecific antibodies that comprise a binding domain from any of the mAbs mentioned above, preferably VE1 or A2T or A2B or mAbs with the same epitope as VE1 or A2T or A2B, or having CDRs from VE1 or A2T or A2B, including humanized forms of VE1 or A2T or A2B such as HuVE1 or HuA2T, are encompassed in the invention. A second binding domain of such a bispecific antibody may for example bind to another growth factor such as epidermal growth factor (EGF), any of the fibroblast growth factors such as FGF2, hepatocyte growth factor (HGF), tumor necrosis factor (TNF), transforming growth factor beta (TGF-β1, TGF-β2, or TGF-β3), any form of platelet derived growth factor (PDGF) or neuregulin or heregulin, and angiopoietin 1 or 2, or alternatively any extracellular domains of any receptor for these growth factors. Preferably the second binding domain will be from a humanized or human mAb. Binding to human forms of these growth factors or receptors is preferred. Examplary sequences of these growth factors and receptors are readily available from e.g., the Swiss-Prot database. The binding (variable) domain of the anti-HGF mAb HuL2G7 described in U.S. Pat. No. 7,632,926 (which is herein incorporated by reference for all purposes), or a binding domain comprising one or more of its CDRs, is especially preferred, as is the binding domain of humanized forms of the anti-FGF2 mAb GAL-F2 (sequences shown in FIG. 11 of U.S. Pat. No. 8,101,725). In particularly preferred embodiments, one binding domain is from any of the anti-VEGF mAbs disclosed herein such as HuVE1, and a second binding domain is from any of the anti-Ang2 mAbs disclosed herein such as HuA2T.

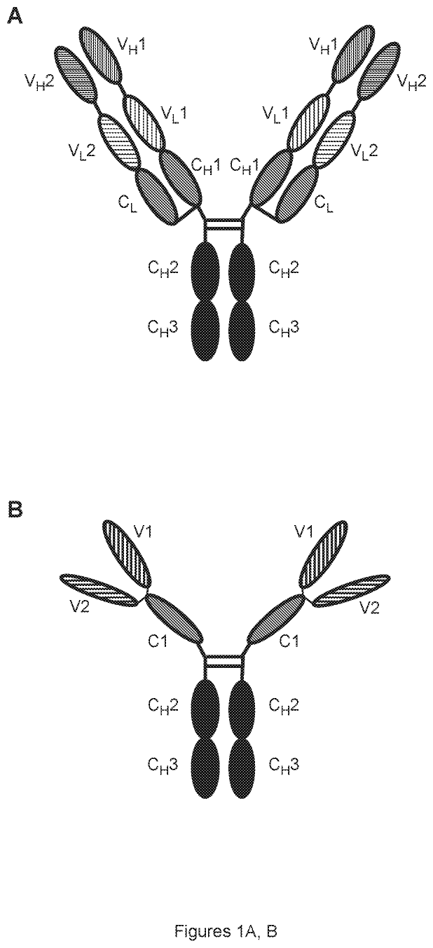

The bispecific antibody of the invention may be in any format, such as any of those listed in Kontermann, op. cit. In one preferred embodiment, the bispecific antibody is in the Bs(scFv)4-IgG format described in Zuo et al., op. cit. and illustrated in FIG. 1. In this format, one binding domain in single chain (scFv) form is connected to the CL region and thus becomes the N-terminal domain of the light chain, while the other binding domain in scFv form is connected to the C_{H}1 domain and thus becomes the N-terminal domain of the heavy chain; two light chains and two heavy chains form a homodimer as in an ordinary IgG antibody, but containing two of each binding domain. Thus, an advantage of the Bs(scFv)4-IgG format is that it is a homodimer, with the same heavy chain and light chain in each monomer, so that no precautions need to be taken to ensure correct heterodimerization. The linker within each scFv connecting the V_{L} and V_{H} regions is often chosen as (G₄S)₃GS. Each scFv binding domain may be in the form V_{L}-linker-V_{H} or in the form V_{H}-linker-V_{L} (as shown in FIG. 1A), and either binding domain may be part of the light chain while the other is part of the heavy chain, so in total 2×2×2=8 variants of a Bs(scFv)4-IgG antibody can be made from two given binding domains (e.g., those of HuVE1 and HuL2G7 or HuA2T), which may have differing properties. In especially preferred embodiments of the invention, the HuVE1 V domain in the scFv V_{H}-linker-V_{L} form is connected to C_{H}1, while the other antibody domain such as the HuL2G7 or HuA2T V domain in the scFv V_{H}-linker-V_{L} form is connected to C_{L}.

In another embodiment of the invention, the bispecific antibody is in the Double Variable Domain format described in, e.g., Wu et al., op. cit., (see FIG. 1A with labeling therein). Such a bispecific mAb contains two of each of the binding domains, with one of each binding domain linked in sequence. A variety of peptide linkers may be used to connect the first and second domains, e.g., ASTKGPSVFPLAP in the heavy chain and RTVAAPSVIFIPP in the light chain, or (G₄S)₃GS in both chains. For example, the variable domain of HuL2G7 or HuA2T could be the first domain (VL1-VH1), while the variable domain of HuVE1 could be the second domain (VL2-VH2); and the linkers could be the former ones mentioned above.

In other preferred embodiments of the invention, one monomer of the HuVE1 mAb comprising a light and heavy chain pairs with one monomer of the HuL2G7 or HuA2T mAb comprising a light and heavy chain to form a heterodimer with the normal configuration of an IgG molecule. If all four chains are to be expressed in a cell, formation of the desired heterodimer bispecific antibodies instead of homodimers is promoted by inserting knobs and holes into the CH3 regions of the respective heavy chains (Ridgway et al., Protein Eng 9:617-21, 1996; Atwell et al., J Mol Biol 270:26-35, 1997; and U.S. Pat. No. 7,695,936), while correct pairing of the light and heavy chains to form each HuVE1 and HuL2G7 or HuA2T monomer is promoted by “crossing over” of heavy chain and light chain domains within one of the monomers (Schaefer et al., Proc Natl Acad Sci USA 108:11187-92, 2011; WO 2009/080251; WO 2009/080252; WO 2009/080253).

The invention provides also variant bispecific antibodies whose light and heavy chain differ from the ones specifically described above by a small number (e.g., typically no more than 1, 2, 3, 5 or 10) of replacements, deletions or insertions, usually in the C region or V region framework but possibly in the CDRs. Most often the replacements made in the variant sequences are conservative with respect to the replaced amino acids. Amino acids can be grouped as follows for determining conservative substitutions, i.e., substitutions within a group: Group I (hydrophobic sidechains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe.

Preferably, replacements in the bispecific antibody have no substantial effect on the binding affinity or potency of the antibody, that is, on its ability to neutralize the biological activities of VEGF and the target of the second binding domain such as HGF or Ang-2. Preferably the variant sequences are at least 90%, more preferably at least 95%, and most preferably at least 98% identical to the original sequences. In addition, other allotypes or isotypes of the constant regions may be used.

### 4. Therapeutic Methods

In a preferred embodiment, the present invention provides a pharmaceutical formulation comprising an antibody described herein. Pharmaceutical formulations contain the mAb in a physiologically acceptable carrier, optionally with excipients or stabilizers, in the form of lyophilized or aqueous solutions. Acceptable carriers, excipients or stabilizers are nontoxic to recipients at the dosages and concentrations employed, and include buffers such as phosphate, citrate, or acetate at a pH typically of 5.0 to 8.0, most often 6.0 to 7.0; salts such as sodium chloride, potassium chloride, etc. to make isotonic; antioxidants, preservatives, low molecular weight polypeptides, proteins, hydrophilic polymers such as polysorbate 80, amino acids, carbohydrates, chelating agents, sugars, and other standard ingredients known to those skilled in the art (Remington's Pharmaceutical Science 16^{th} edition, Osol, A. Ed. 1980). The mAb is typically present at a concentration of 1-100 mg/ml, but most often 10-50 mg/ml, e.g., 10, 20, 30, 40 or 50 mg/ml.

In another preferred embodiment, the invention provides a method of treating a patient with a disease by administering an anti-VEGF or Anti-Ang-2 mAb of the invention such as VE1 or A2T or their humanized and/or bispecific forms in a pharmaceutical formulation, typically in order to inhibit angiogenesis associated with the disease. The mAb prepared in a pharmaceutical formulation can be administered to a patient by any suitable route, especially parentally by intravenous infusion or bolus injection, intramuscularly or subcutaneously. Intravenous infusion can be given over as little as 15 minutes, but more often for 30 minutes, or over 1, 2 or even 3 hours. The mAb can also be injected directly into the site of disease (e.g., a tumor), or encapsulated into carrying agents such as liposomes. The dose given is sufficient to alleviate the condition being treated (“therapeutically effective dose”) and is likely to be 0.1 to 5 mg/kg body weight, for example 1, 2, 3, 4 or 5 mg/kg, but may be as high as 10 mg/kg or even 15 or 20 or 30 mg/kg, e.g., in the ranges 1-10 mg/kg or 1-20 mg/kg. A fixed unit dose may also be given, for example, 50, 100, 200, 500 or 1000 mg, or the dose may be based on the patient's surface area, e.g., 1000 mg/m². Usually between 1 and 8 doses, (e.g., 1, 2, 3, 4, 5, 6, 7 or 8) are administered to treat cancer, but 10, 20 or more doses may be given. The mAb can be administered daily, biweekly, weekly, every other week, monthly or at some other interval, depending, e.g. on the half-life of the mAb, for 1 week, 2 weeks, 4 weeks, 6 weeks, 8 weeks, 3-6 months or longer. Repeated courses of treatment are also possible, as is chronic administration.

Diseases especially susceptible to therapy with the anti-VEGF and/or Anti-Ang-2 mAbs of this invention include those associated with angiogenesis and/or elevated levels of VEGF and/or Ang-2, including solid tumors, for example ovarian cancer, breast cancer, lung cancer (small cell or non-small cell), colon cancer, prostate cancer, pancreatic cancer, gastric cancer, liver cancer (hepatocellular carcinoma), kidney cancer (renal cell carcinoma), head-and-neck tumors, melanoma, sarcomas, and brain tumors (e.g., glioblastomas). Hematologic malignancies such as leukemias and lymphomas may also be susceptible. In a preferred embodiment, the mAb is administered in combination with (i.e., together with, that is, before, during or after) other therapy. For example, to treat cancer, the mAb of this invention may be administered together with any one or more of the known chemotherapeutic drugs, for example alkylating agents such as carmustine, chlorambucil, cisplatin, carboplatin, oxaliplatin, procarbazine, and cyclophosphamide; antimetabolites such as fluorouracil, floxuridine, fludarabine, gemcitabine, methotrexate and hydroxyurea; natural products including plant alkaloids and antibiotics such as bleomycin, doxorubicin, daunorubicin, idarubicin, etoposide, mitomycin, mitoxantrone, vinblastine, vincristine, and Taxol (paclitaxel) or related compounds such as Taxotere®; the topoisomerase 1 inhibitor irinotecan; and inhibitors of tyrosine kinases such as Gleevec® (imatinib), Sutent® (sunitinib), Nexavar® (sorafenib), Tarceva® (erlotinib), Tykerb® (lapatinib), Iressa® (gefitinib) and Xalkori® (crizotinib); Rapamycin® (sirolimus) and other mTOR inhibitors; and inhibitors of angiogenesis; and all approved and experimental anti-cancer agents listed in WO 2005/017107 A2 (which is herein incorporated by reference). The antibody of this invention may be used in combination with 1, 2, 3 or more of these other agents, preferably in a standard chemotherapeutic regimen. Normally, the other agents are those already believed or known to be effective for the type of cancer being treated.

Other agents with which the anti-VEGF and/or Anti-Ang-2 mAbs of this invention can be administered to treat cancer include biologics such as monoclonal antibodies, including Herceptin® or Perjeta® (pertuzumab), against the HER2 antigen; Avastin® against VEGF; or antibodies to the Epidermal Growth Factor (EGF) receptor such as Erbitux® (cetuximab) and Vectibix® (panitumumab), as well as antibody-drug conjugates such as Kadcyla™ (ado-trastuzumab emtansine). MAbs against HGF are especially preferred for use with the anti-VEGF or anti-Ang-2 mAb, including mAb L2G7 (Kim et al., Clin Cancer Res 12:1292, 2006 and U.S. Pat. No. 7,220,410) and particularly its chimeric and humanized forms such as HuL2G7 (U.S. Pat. No. 7,632,926); the human anti-HGF mAbs described in WO 2005/017107 A2, particularly 2.12.1; and the HGF binding proteins described in WO 07143090 A2 or WO 07143098 A2; and other neutralizing anti-HGF mAbs that compete for binding with any of the aforementioned mAbs. MAbs that bind to RON or to the Met receptor of HGF are also preferred, for example the anti-cMet mAb OA-5D5 (Martens et al., Clin Cancer Res 12:6144, 2006) that has been genetically engineered to have only one “arm”, i.e. binding domain. Mabs that bind to FGF2 such as humanized forms of GAL-F2 as disclosed in U.S. Pat. No. 8,101,725 are also preferred. Moreover, the anti-VEGF or Anti-Ang-2 mAb can be used together with any form of surgery and/or radiation therapy.

Treatment (e.g., standard chemotherapy) including the anti-VEGF and/or Anti-Ang-2 mAb of this invention antibody may increase the median progression-free survival or overall survival time of patients with a particular type of cancer such as those listed above by at least 20% or 30% or 40% but preferably 50%, 60% to 70% or even 100% or longer, compared to the same treatment (e.g., chemotherapy) but without mAb; or by (at least) 2, 3, 4, 6 or 12 months. In addition or alternatively, treatment (e.g., standard chemotherapy) including the mAb may increase the complete response rate, partial response rate, or objective response rate (complete+partial) of patients (especially when relapsed or refractory) by at least 30% or 40% but preferably 50%, 60% to 70% or even 100% compared to the same treatment (e.g., chemotherapy) but without the anti-VEGF mAb.

Typically, in a clinical trial (e.g., a phase II, phase II/111 or phase III trial), the aforementioned increases in median progression-free or overall survival and/or response rate of the patients treated with chemotherapy plus the anti-VEGF and/or Anti-Ang-2 mAb of this invention, relative to the control group of patients receiving chemotherapy alone (or plus placebo), is statistically significant, for example at the p=0.05 or 0.01 or even 0.001 level. It is also understood that response rates are determined by objective criteria commonly used in clinical trials for cancer, e.g., as accepted by the National Cancer Institute and/or Food and Drug Administration, for example the RECIST criteria (Response Evaluation Criteria In Solid Tumors).

The anti-VEGF and/or Anti-Ang-2 mAbs of this invention may also be used to treat endometriosis and inflammatory and autoimmune diseases, especially those associated with angiogenesis or VEGF or Ang-2, including inflammatory bowel disease (Crohn's disease and ulcerative colitis) in which a role for VEGF has been shown (see Gorlatova et al., PLoS One 6:e27269, 2011 and Hauser et al., Genes Immun 13:321-7, 2012), rheumatoid arthritis, psoriasis, and kidney disease such as glomerulonephritis, as well as eye diseases such as age-related macular degeneration or diabetes-associated retinopathy. For eye diseases, a fragment of the mAb such as an Fab or (Fab′)2 that can be injected directly into the eye may be especially suitable.

### 5. Other Methods

The mAbs of the invention also find use in diagnostic, prognostic and laboratory methods. They may be used to measure the level of VEGF or Ang-2 in a tumor or in the circulation of a patient with a tumor, and therefore to follow and guide treatment of the tumor. For example, a tumor associated with elevated or high levels of VEGF (respectively Ang-2) would be especially susceptible to treatment with an anti-VEGF (respectively Anti-Ang-2) mAb. In particular embodiments, the mAbs can be used in an ELISA or radioimmunoassay to measure the level of VEGF or Ang-2, e.g., in a tumor biopsy specimen or in serum or in media supernatant of VEGF-secreting cells in cell culture. The use of two anti-VEGF (respectively anti-Ang-2) mAbs binding to different epitopes (i.e., not competing for binding) is especially useful in developing a sensitive “sandwich” ELISA to detect VEGF (respectively Ang-2). For various assays, the mAb may be labeled with fluorescent molecules, spin-labeled molecules, enzymes or radioisotopes, and may be provided in the form of kit with all the necessary reagents to perform the assay for VEGF or Ang-2. In other uses, the anti-VEGF (respectively anti-Ang-2) mAbs are used to purify VEGF (respectively Ang-2) by affinity chromatography.

### 6. Examples

### Example 1: Generation of Anti-VEGF mAbs

To generate and assay mAbs that bind to and block the activities of human VEGF, a glutathione synthetase—VEGF fusion protein, GST-VEGF, was first produced. For this purpose, cDNA encoding full length human VEGF165 was constructed and inserted into a derivative of the pGEX expression vector (Invitrogen), and transformed and expressed in BL21(DE3) E. coli cells (Novagen), using standard methods of molecular biology. GST-VEGF was purified from E. coli lysate by using a glutathione-agarose column (Sigma-Aldrich). Two other fusion proteins, VEGF-FLAG (respectively FLAG-VEGF) were produced by linking a FLAG tag (amino acids DYKDDDDK) to the carboxy (resp. amino) terminus of human VEGF165 in a derivative of the pCI vector (Invitrogen), and expressing in mammalian 293F cells. The amount of VEGF-FLAG or FLAG-VEGF secreted in the culture fluid was quantitated using a VEGF specific ELISA. For blocking assays, the extracellular domain of the human VEGF receptor 2 (VEGFR2) (amino acids 1 to 760) was linked to the human Ig gamma-1 Fc constant region (hinge-cH2-cH3) to generate human VEGFR-Fc, which was produced in mammalian cells and purified using a protein A column. Human VEGF-121, VEGF-165, VEGF-186, VEGF-B, VEGF-C, VEGF-D, VEGFR1 and mouse VEGF-A were purchased (R&D Systems).

Balb/c mice were immunized in each hind footpad twice weekly 16-18 times with purified GST-VEGF in Ribi adjuvant (10 μg for the first injection and 5 μg for subsequent injections). Three days after the final boost, popliteal lymph node cells were fused with murine myeloma cells, P3X63AgU.1 (ATCC CRL 1597), using 35% polyethylene glycol. Hybridomas were selected in HAT medium as described (Chuntharapai and Kim, J Immunol 163:766, 1997). Ten days after the fusion, hybridoma culture supernatants were screened in a VEGF binding ELISA followed by the VEGF/VEGFR blocking ELISA described below. Selected hybridomas were cloned twice by screening for VEGF binding as well as for VEGF/VEGFR blocking. After screening approximately 20,000 hybridomas from 13 fusions, VE1.7 was chosen as the best anti-VEGF antibody. This antibody will be designated VE1 herein. The isotype of VE1 was determined to be IgG2a, kappa using an isotyping kit

### Example 2: Assays Used to Characterize Anti-VEGF mAbs

Each step of each ELISA assay described in this patent application was performed by room temperature incubation with the appropriate reagent for 1 hour, except the initial plate coating step(s) was done overnight at 4° C., followed by blocking with 2% BSA for 1 hr. Between each step, plates were washed 3 times in PBS containing 0.05% Tween 20. Data points were generally in triplicate; there was generally little variability between triplicate data points. To measure direct binding of mAbs to VEGF, plates were first coated with heparin (50 μg/ml) overnight, followed by incubation with human VEGF165 (0.3 μg/ml) overnight, and then blocked with BSA. Wells were incubated with hybridoma supernatant for screening or with increasing concentrations of purified VE1 mAb or other anti-VEGF mAb to be tested, and the bound mAb was detected by addition of HRP-goat anti-mouse IgG and then TMB substrate. To measure the ability of mAbs to bind to VEGF in solution (capture assay), plates were first coated with goat anti-mlgG-Fc (2 μg/ml). Wells were incubated with increasing concentrations of purified VE1 mAb or other anti-VEGF mAb to be tested and then with VEGF-Flag (0.5 μg/ml) for purified mAbs or VEGF-FLAG+FLAG-VEGF for hybridoma supernatant, plus mouse IgG (30 μg/ml). The bound VEGF-Flag was detected by the addition of HRP-anti-Flag M2 (Sigma) in the presence of mouse IgG (15 μg/ml) and then TMB substrate. To measure blocking activity of mAbs, plates were first coated with goat anti-hlgG-Fc (2 μg/ml). Wells were then incubated with VEGFR-Fc (0.5 μg/ml), and then with hybridoma supernatant for screening or with increasing concentrations of purified VE1 mAb or other anti-VEGF mAb to be tested, premixed with VEGF-Flag (0.5 μg/m). The bound VEGF-Flag was detected by the addition of HRP-anti-Flag M2 followed by TMB substrate.

### Example 3: Binding and Blocking Activity of VE1 Antibody

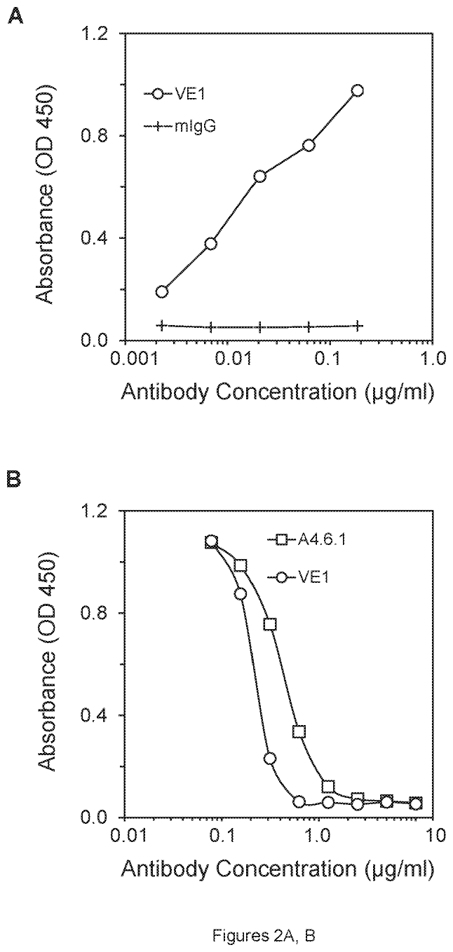

The ability of VE1 to bind to VEGF was demonstrated in the direct binding and capture assays described above (FIG. 2A). The ability of VE1 to inhibit binding of VEGF to its receptor VEGFR (VEGFR2), a key property of a neutralizing anti-VEGF mAb, was compared with that of the A4.6.1 mAb which was humanized to make bevacizumab, using the blocking assay described above. As shown in FIG. 2B, VE1 inhibited binding of VEGF to VEGFR completely, and at substantially lower concentrations than A4.6.1.

### Example 4: Construction and Characterization of Humanized VE1 Antibodies

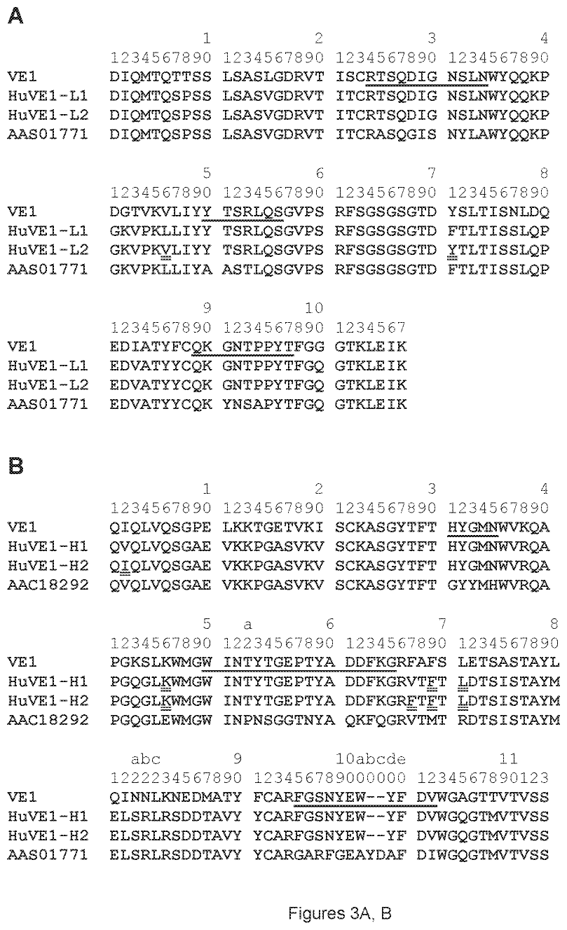

Cloning of the light and heavy chain variable regions of the VE1 mAb, construction and expression of a chimeric mAb, and design, construction, expression and purification of a humanized VE1 mAb were all performed using standard methods of molecular biology, e.g. as described in U.S. Pat. No. 7,632,926 for the L2G7 mAb, which is herein incorporated by reference for all purposes. The amino acid sequences of the (mature) light and heavy chain variable (V) regions of VE1 are shown respectively in FIGS. 3A and 3B, top lines labeled VE1. More specifically, to design a humanized VE1 mAb, the methods of Queen et al., U.S. Pat. Nos. 5,530,101 and 5,585,089 were generally followed. The human VK sequence AAS01771 and VH sequence AAC18292, as shown respectively in FIGS. 3A and 3B, bottom lines, were respectively chosen to serve as acceptor sequences for the VE1 VL and VH sequences because they have particularly high framework homology (i.e., sequence identity) to them. A computer-generated molecular model of the VE1 variable domain was used to locate the amino acids in the VE1 framework that are close enough to the CDRs to potentially interact with them. To design the humanized VE1 light and heavy chain variable regions, the CDRs from the mouse VE1 mAb were first conceptually grafted into the acceptor framework regions. At framework positions where the computer model suggested significant contact with the CDRs, which may be needed to maintain the CDR conformation, the amino acids from the mouse antibody were substituted for the human framework amino acids. Two versions of each of the humanized light chain and humanized heavy chain were designed in this manner. For the light chain, either no such substitutions were made (HuVE1-L1), or substitutions were made at residues 46 and 81 (HuVE1-L2); for the heavy chain, residues 46, 69 and 71 of the heavy chain were substituted (HuVE1-H1) or these residues plus the additional residues 2 and 67 were substituted (HuVE1-H2), all with reference to Kabat numbering. These humanized light and heavy chain V region sequences are shown in FIGS. 3A and 3B respectively, middle lines as labeled, where they are aligned against the respective VE1 donor and human acceptor V regions—the CDRs (as defined by Kabat) are underlined and the substituted amino acids listed above are double-underlined. The V region sequences were linked with human kappa and gamma-1 C regions. By combining each of the humanized light chains with each of the humanized heavy chains, four different humanized VE1 antibodies designated HuVE1 #1, #2, #3 and #4 were made, as shown in the following table, where the number of substitutions in each chain is given in parentheses. In addition, a chimeric VE1 mAb designated ChVE1 was constructed by combining the V regions of (mouse) VE1 with human kappa and gamma-1 C regions.

TABLE
HuVE1 Variants

| HuVE1 | Light Chain | Heavy Chain |
|---|---|---|
| #1 | L1 (0) | H1 (3) |
| #2 | L1 (0) | H2 (5) |
| #3 | L2 (2) | H1 (3) |
| #4 | L2 (2) | H2 (5) |

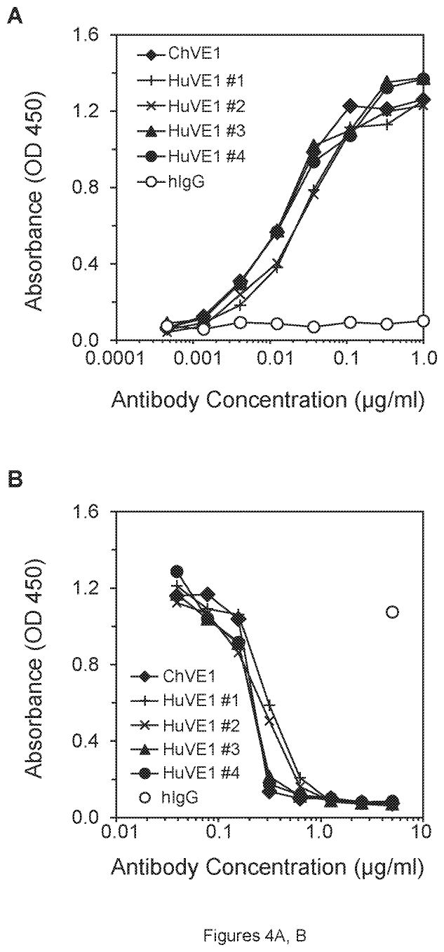

The ability of ChVE1 and the four versions of HuVE1 to bind to VEGF were compared in a capture assay as described above in Example 2, but with goat anti-hlgG-Fc instead of anti-mlgG-Fc used to bind the mAbs to the plate. ChVE1 rather than VE1 was used so that all the mAbs could be compared in one assay using the same reagents; ChVE1 is expected to bind the same as VE1 because it has the same V regions. As seen in FIG. 4A, all the antibodies bound well to VEGF, but HuVE1 #3 and #4 bound about as well as ChVE1, whereas HuVE1 #1 and HuVE1 #2 did not bind quite as well. The ability of ChVE1 and the four versions of HuVE1 to block the binding of VEGF to VEGFR were compared in the assay described above in Example 2. As seen in FIG. 4B, all the antibodies blocked binding of VEGF to VEGFR, but HuVE1 #3 and #4 inhibited about as well as ChVE1, whereas HUVE1 #1 and HuVE1 #2 did not inhibit quite as well. These results show that the two amino acid substitutions made in HuVE1-L2 improved the activity of the humanized mAbs containing this light chain, and that no affinity was lost when humanizing VE1 provided HuVE1-L2 was used for the light chain. Further studies were conducted primarily with HuVE1 #4, which will be designated HuVE1 in what follows.

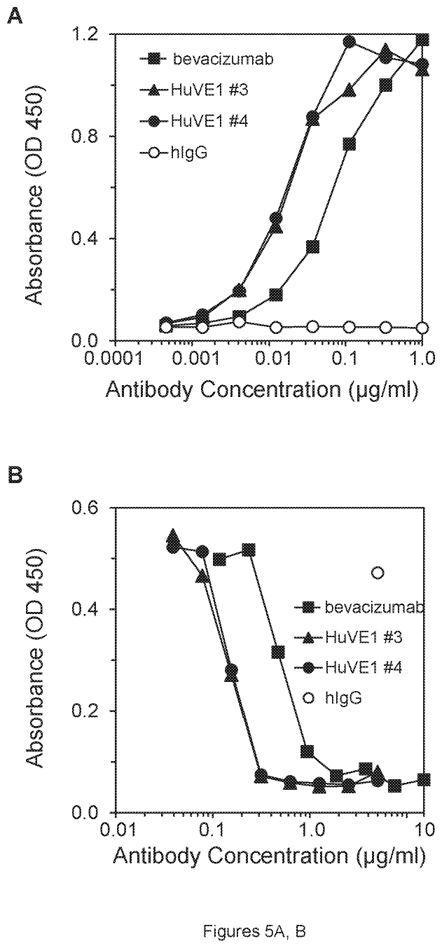

The ability of HuVE1 #3 and HuVE1 #4 to bind to (capture) VEGF and to block binding of VEGF to VEGFR were compared with bevacizumab in the same assays used above, as shown for binding in FIG. 5A and blocking in FIG. 5B. Using software, the Effective Concentration 50% (EC50) for binding was calculated from this data as 0.09 μg/mL for bevacizumab but only 0.02 μg/mL for both HuVE1 #3 and HuVE1 #4. Similarly, the Inhibitory Concentration 50% (IC50) for blocking was calculated as 0.34 μg/mL for bevacizumab but only 0.05 μg/mL for HuVE1 #3 and 0.06 μg/mL for HuVE1 #4, so that in the critical activity of inhibiting binding of VEGF to VEGFR, HuVE1 #3 and HuVE1 #4 were respectively about 7-fold and 6-fold more potent than bevacizumab.

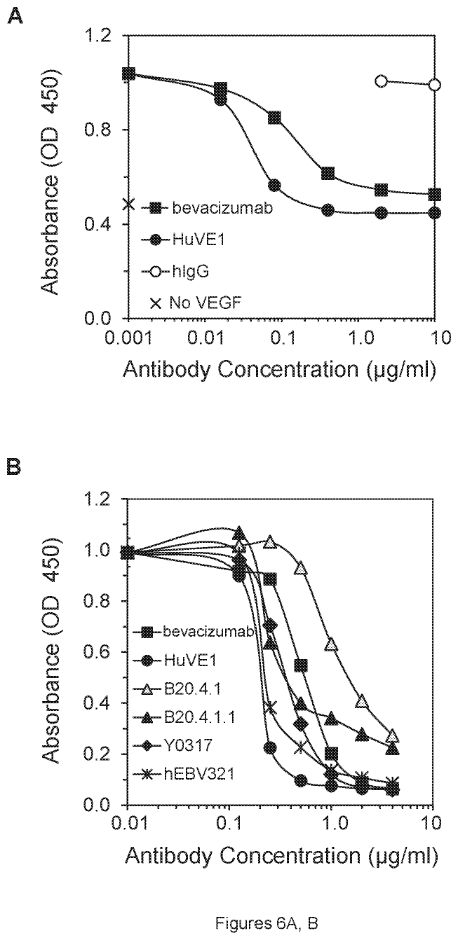

Finally the ability of HuVE1 (HuVE1 #4) to inhibit VEGF-induced proliferation of human umbilical vascular endothelial cells (HUVEC), an assay for neutralizing activity of the mAb, was determined in comparision to bevacizumab. To perform this assay, 5,000 HUVECs were plated per well of a 96-well ELISA plate in EBM-2 medium with 1% FCS and 0.1% BSA and incubated overnight, followed by incubation in EBM-2 with 0.1% FCS and 0.1% BSA for 24 hr The cells were then incubated in the same medium with 20 ng/mL VEGF plus increasing concentrations of the mAbs for 3 days; the extent of proliferation was determined using WST-8 according to the manufacturer's directions. As seen in FIG. 6A, HuVE1 was able to inhibit proliferation to background level (no VEGF) with an IC50 computed as 0.057 μg/mL compared to 0.36 μg/mL for bevacizumab, i.e., HuVE1 was about 6-fold more potent than bevacizumab in this bioassay, fully consistent with the above result in the receptor blocking assay.

We also compared the activity of HuVE1 with that of several previously published anti-VEGF mAbs claimed to have high binding affinity or activity, as measured by the ability to inhibit binding of VEGF to VEGFR2 in the assay described above. The other mAbs were first synthesized based on their published sequences: the humanized rabbit mAb hEBV321 (US 2012/0231011), the affinity-matured humanized mAb Y0317 (EP 1 787 999), and the human mAbs B20.4.1 and B20.4.1.1 (US 2009/0142343). As seen from FIG. 6B, none of these mAbs were as active as HuVE1 in the assay, and some were notably less active.

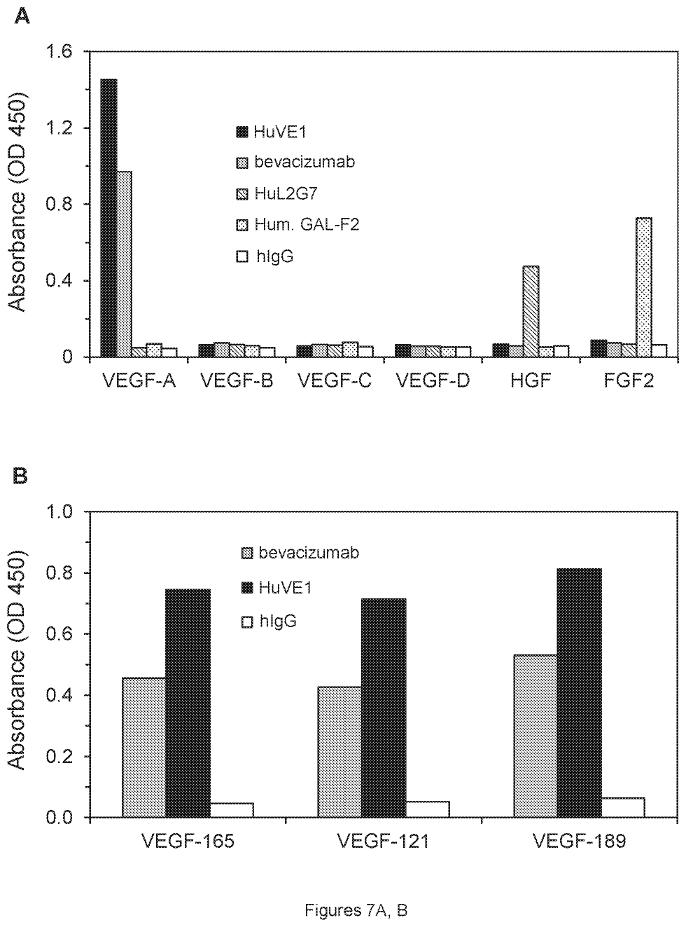

To show that HuVE1 specifically binds VEGF-A, 0.2 μg/mL of that protein as well as VEGF-B, VEGF-C, VEGF-D and two other growth factors, HGF and FGF2, were first incubated on ELISA plates that had been coated with heparin (50 μg/mL). Then the wells were incubated with 2 μg/mL of HuVE1 or the control mAbs bevacizumab, HuL2G7, humanized GAL-F2 anti-FGF2, or negative control hlgG, followed by detection with HRP-goat anti-human IgG and then TMB substrate. As seen in FIG. 7A, the control mAbs HuL2G7 and humanized GAL-F2 respectively bound only to HGF and FGF2, while HuVE1 (and bevacizumab) bound only to VEGF-A above background level (binding of hlgG), showing the specificity of HuVE1 for VEGF-A. Since (mouse) VE1 binds in the same way as its humanized form, it must also be specific for VEGF-A. In another experiment conducted in a similar manner, but coating the ELISA plate with A4.6.1 (2 μg/mL) rather than heparin to capture VEGF, HuVE1 (and bevacizumab) bound to three different isoforms of VEGF-A (FIG. 7B): the shortest form VEGF₁₂₁, the most abundant form VEGF₁₆₅, and VEGF₁₈₉.

### Example 5: Epitope of HuVE1

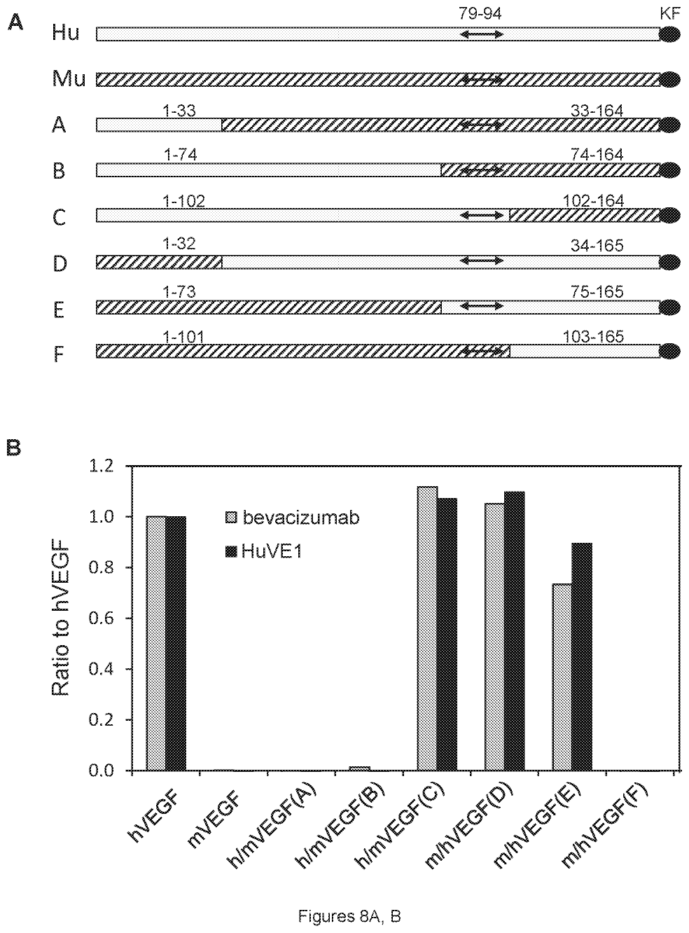

To determine the epitope of HuVE1 (and therefore VE1), we measured its ability to bind to a series of derivatives of VEGF linked at the carboxy end to the human kappa constant region followed by Flag peptide (VEGF-KF). For this purpose ELISA wells were coated with goat anti-human-Ig-Fc (2 μg/mL), blocked with 2% BSA, incubated with 0.1 μg/mL HuVE1 (HuVE1 #4) or for comparison bevacizumab, followed by the appropriate form of VEGF-KF, and detected with HRP-M2-anti-Flag and substrate. It was first noted that HuVE1 and bevacizumab do not bind to mouse VEGF (second column of FIG. 8B as labeled). Following an approach widely used in the art, we thus made a series of chimeric molecules between human VEGF and mouse VEGF (FIG. 8A) and measured the binding of HuVE1 and bevacizumab to each. As seen in FIG. 8B, these mAbs bound to only those chimeric VEGFs in which the amino acid region 79-104 (shown by a short double arrow in FIG. 8A) came from human VEGF, thus identifying this region as containing the epitopes of the mAbs, consistent with a previous report for bevacizumab.

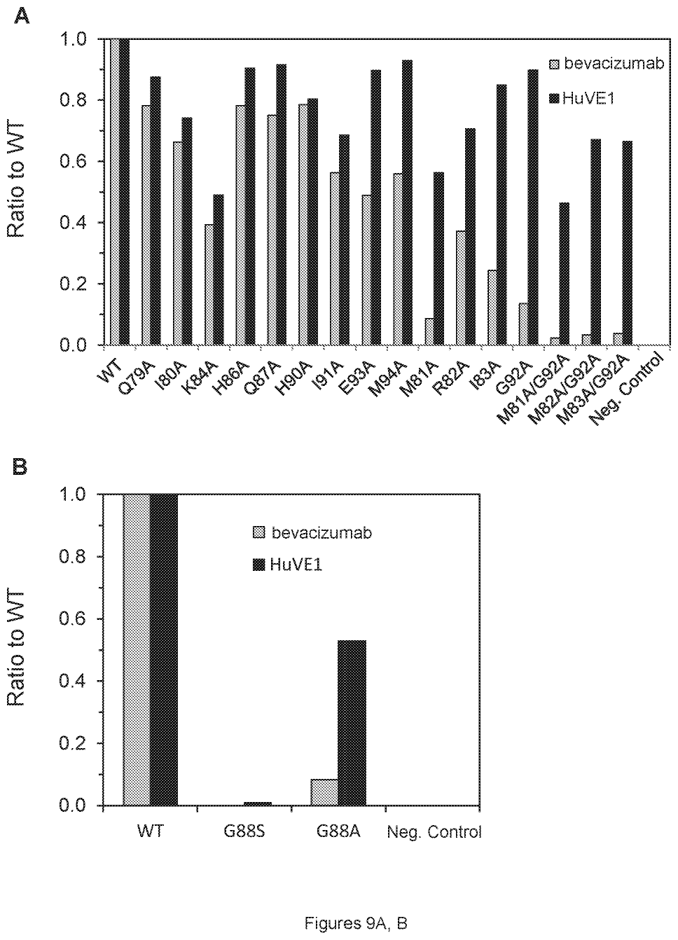

To more precisely compare the epitope of HuVE1 with that of bevacizumab, binding of these mAbs to a series of mutants of VEGF were measured (FIG. 9). Certain mutations such as M81A and K84A (FIG. 9A) and G88S or G88A (FIG. 9B) substantially reduced the binding of both HuVE1 and bevacizumab, indicating that amino acid positions 81, 84, and 88 are in the epitopes of both these mAbs. However, mutations at amino acids such as 83 and 92 substantially reduced binding of bevacizumab but had little or no effect on binding of HuVE1. This is even more clearly seen with the double mutation M83A/G92A, which essentially eliminated binding of bevacizumab but had at most a modest effect on binding of HuVE1. We conclude that certain amino acids such as M83 and G92 are in the epitope of bevacizumab but not HuVE1, so these mAbs must have overlapping but not identical epitopes on VEGF.

### Example 6: Generation of Anti-Ang-2 mAbs

To generate and assay mAbs that bind to and block the activities of human Ang-2, several Fc-fusion proteins were constructed using standard methods of molecular biology. For this purpose, cDNAs were constructed encoding the fibrinogen-like (F) domain (amino acids 274 to 496) of human, murine, and murine-human or human-murine chimeric Ang-2 (denoted respectively as hAng-2(F), mAng-2(F), m/hAng-2(F) and h/mAng-2(F)), with the chimeric forms respectively consisting of amino acids 274-410 of murine Ang-2 linked to amino acids 411-496 of human Ang-2 or vice versa. These cDNAs were linked to the human Ig gamma-1 Fc region (hinge-cH2-cH3) either at the N-terminus or C-terminus (denoted respectively Fc-Ang-2(F) and Ang-2(F)-Fc, with appropriate modifiers), or at the C-terminus to the human kappa constant region followed by the Flag peptide (denoted hAng-2(F)-KF, etc.), inserted into derivatives of the pCI vector (Invitrogen), transfected and expressed in 293F mammalian cells. The Fc fusion proteins were purified from 293F culture supernatant by using a protein A column (Sigma-Aldrich). Another fusion protein, Flag-m/hAng-2(F) was produced by linking a FLAG tag (amino acids DYKDDDDK) to the N-terminus of murine/human chimeric Ang-2(F) in a derivative of the pCI vector (Invitrogen), expression in 293F cells and purification using an anti-FLAG column. Another protein, Peptide-KLH, was made by chemically conjugating a peptide from hAng-2 (amino acids 464-483) to KLH. For blocking assays, the extracellular domain of the human Tie-2 receptor (amino acids 1 to 760) was linked to the human Ig gamma-1 Fc constant region (hinge-cH2-cH3) to generate human Tie-2-Fc, which was produced in mammalian cells and purified using a protein A column.

Balb/c female mice were immunized in each hind footpad twice weekly with antigen in Ribi adjuvant (10 μg for the first injection and 5 μg for subsequent injections or as indicated). One group of mice were immunized 12 times with hAng-2(F)-Fc plus a final boost with Fc-hAng-2(F). A second group of mice were immunized 6 times with hAng-2(F)-Fc alternating with mAng-2(F)-Fc, then 3 times with Flag-m/hAng-2(F), then 3 times with hAng-2(F)-Fc alternating with mAng-2(F)-Fc, then 2 times with Peptide-KLH (6 μg) and a final boost with m/hAng-2(F)-Fc. Three days after this final boost, popliteal lymph node cells were fused with murine myeloma cells P3X63AgU.1 and hybridomas selected in HAT medium as described above. Hybridoma culture supernatants were initially screened in a hAng2(F)-KF capture ELISA followed by a Ang-2Tie2 blocking ELISA as described below. Selected hybridomas were cloned twice by screening for Ang2(F)-KF binding as well as for Ang-2/Tie2 blocking activity. After screening approximately 26,000 hybridomas from 26 fusions, the mAb A2B14.6 (designated here A2B) was selected from a fusion of one of the first group of mice, and the mAb A2T.10.2 (designated here A2T) from a fusion of one of the second group of mice, based on their high binding and blocking activities. A2B and A2T were determined to be respectively of the IgG2b and IgG2a isotypes using an isotyping kit.

To compare A2B and A2T with other anti-Ang-2 mAbs previously shown to have potent anti-angiogenic effects, we synthesized the variable domain genes of several such mAbs based on their published sequences: Ab356 (J. Oliner et al., op. cit; SEQ ID NO. 11 and SEQ ID NO. 12 in WO 03/030833); REGN910 (C. Daly et al., op. cit., REGN910 identified as nesvacumab in the NCI Drug Dictionary at http://www.cancer.gov/publications/dictionaries/cancer-drug?cdrid=693224, and then the sequences obtained at http://www.genome.jp/dbget by search for nesvacumab); MEDI-3167 (A. Buchanan et al., op. cit., FIG. 1A and abstract and text), and LCO6 (M. Thomas et al., op. cit., and S. Fenn et al., Plos One 8: e61953-e61953, 2013; 4IMK in the Protein Data Bank). We also generated human-mouse chimeric antibodies muAb356, muMEDI-3167, and muREGN910, in which the human V regions of the respective mAbs were linked to a mouse C region using standard methods for construction and expression, so these mAbs could be compared to the mouse antibodies A2B and A2T in the same assays; of course the chimeric mAbs are expected to have the same binding and blocking activity as the respective human mAbs.

### Example 8: Characterization of Anti-Ang-2 mAbs

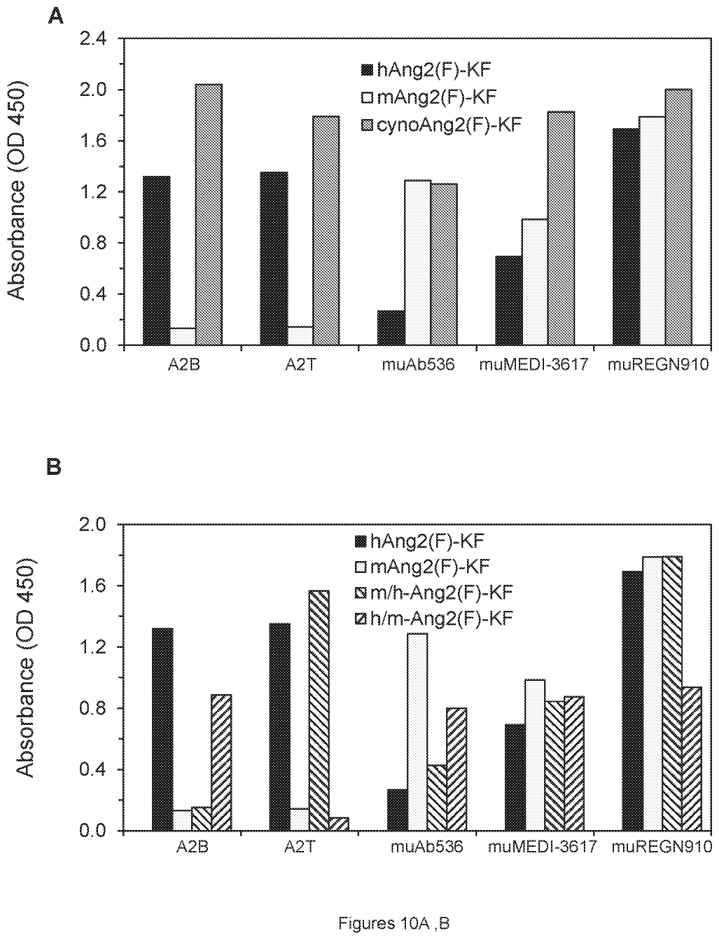

To measure the ability of the anti-Ang-2 mAbs to bind Ang-2, plates coated with goat anti-mouse IgG-Fc (2 μg/mL) were incubated with hybridoma supernatant or purified mAb to be tested (2 μg/mL) followed by hAng-2(F)-KF (1 μg/mL). The bound hAng2(F)-KF was detected by the addition of HRP-goat-anti-IgG-kappa (Sigma) and then TMB substrate. The binding of mAbs to murine Ang-2 and to cynomologus monkey Ang-2 was also measured in this manner, using the appropriate Ang-2(F)-KF constructs. Both mAbs A2B and A2T bind to human and cynomolgus Ang-2, but do not detectably bind to murine Ang-2, unlike the previously published antibodies Ab536, MEDI-3167 and REGN910 (FIG. 10A). Because of this, A2B and A2T must have a different epitope than these previous mAbs. In a similar assay, it was shown that none of these mAbs bind to Ang-1.

To determine the epitopes of the anti-Ang-2 mAbs, we also used an ELISA assay to measure the binding ability of these mAbs to the chimeric m/hAng2(F)-KF and h/mAng2(F)-KF proteins described above. The A2B mAb bound to h/mAng-2-KF but not to m/hAng-2-KF, whereas the A2T mAb bound to m/hAng-2-KF but not to h/mAng-2-KF (FIG. 10B), showing that A2B and A2T have different epitopes and that the epitope of A2T is contained in the amino acid 411-496 region.

To measure the ability of A2B and A2T to block binding of (human) Ang-2 to its receptor (human) Tie-2, ELISA plates were first coated with goat anti-hlgG-Fc (2 μg/mL), followed by Tie-2-Fc (0.3 μg/mL) and then with 50 or 100 ng/mL of Ang-2 mixed with hybridoma supernatant or purified anti-Ang-2 mAb. The bound Ang-2 was detected using 0.5 μg/mL of biotinylated anti-Ang-2 antibody (R&D Systems), followed by addition of HRP-strepavidin and TMB substrate. In this assay, both A2B and A2T completely inhibited binding of Ang-2 to Tie-2, slightly more potently than MEDI-3167 and REGN910 and significantly more potently than Ab536 (FIG. 11).

### Example 9: Construction and Characterization of Humanized A2T Antibodies

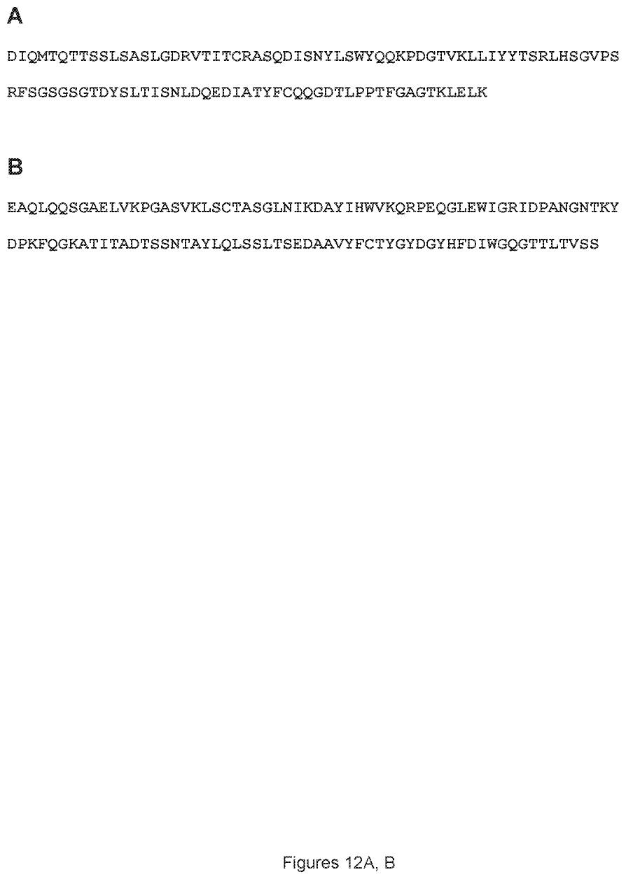

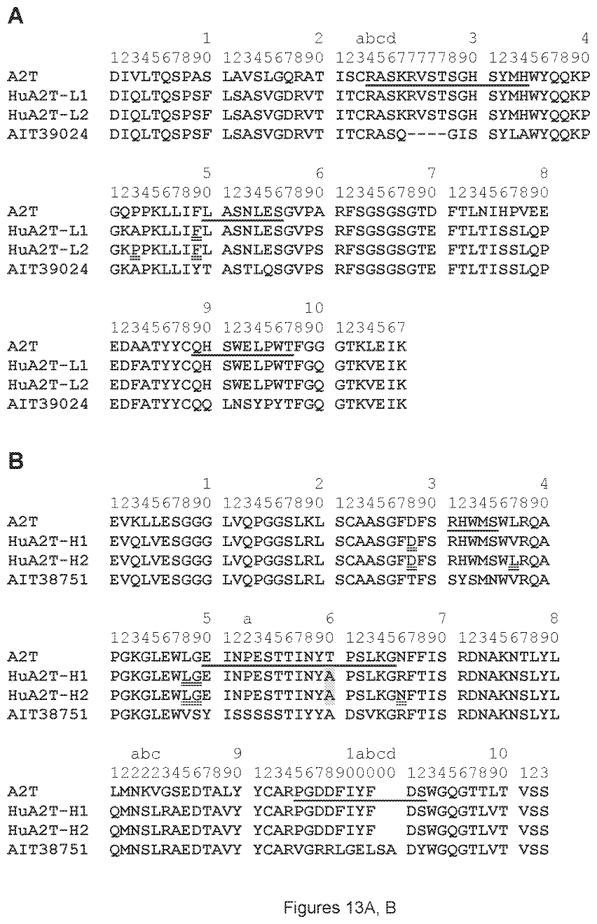

The light and heavy chain variable regions of the A2B and A2T mAbs were cloned and sequenced as described above for VE1—the sequences for A2B are shown in FIG. 12. Construction and expression of a chimeric A2T mAb, and design, construction, expression and purification of humanized A2T mAbs were also all performed using standard methods of molecular biology as described above for the VE1 mAb. The amino acid sequences of the (mature) light and heavy chain variable (V) regions of A2T are shown respectively in FIGS. 13A and 13B, top lines labeled A2T. The human VK sequence AIT39024 and VH sequence AIT38751, as shown respectively in FIGS. 13A and 13B, bottom lines, were respectively chosen to serve as acceptor sequences for the A2T VL and VH sequences because of their high framework homology to them. For the light chain, substitutions from the mouse sequence were made at residue 49 (HuA2T-L1), or at residues 43 and 49 (HuA2T-L2); for the heavy chain, residues 28, 48 and 49 of the heavy chain were substituted (HuA2T-H1) or these residues plus the additional residues 37 and 66 were substituted (HuA2T-H2), all with reference to Kabat numbering. In addition, two versions of each heavy chain were constructed: either with a T at position 60 (in heavy chain CDR2) from the mouse sequence, or with an A at position 60 from the human acceptor sequence in order to eliminate a potential N-linked glycosylation site at position 58 predicted from the pattern N—X—S/T. These humanized light and heavy chain V region sequences are shown in FIGS. 13A and 13B respectively (with the A at position 60 of the heavy chains), middle lines as labeled, where they are aligned against the respective VE1 donor and human acceptor V regions—the CDRs (as defined by Kabat) are underlined and the substituted amino acids listed above are double-underlined. The V region sequences were linked with human kappa and gamma-1 C regions. By combining each of the humanized light chains with each of the humanized heavy chains, two sets of four different humanized A2T antibodies were made, designated HuA2T #1, #2, #3 and #4 with the T at position 60, and respectively HuA2T #1(d), #2(d), #3(d) and #4(d) with the A at position 60, as shown in the following table, where the number of substitutions in each chain is given in parentheses. In addition, a chimeric A2T mAb designated ChA2T was constructed by combining the V regions of (mouse) VE1 with human kappa and gamma-1 C regions.

TABLE
HuA2T Variants

| HuA2T | Light Chain | Heavy Chain |
|---|---|---|
| #1 | L1 (1) | H1 (3) |
| #2 | L1 (1) | H2 (5) |
| #3 | L2 (2) | H1 (3) |
| #4 | L2 (2) | H2 (5) |

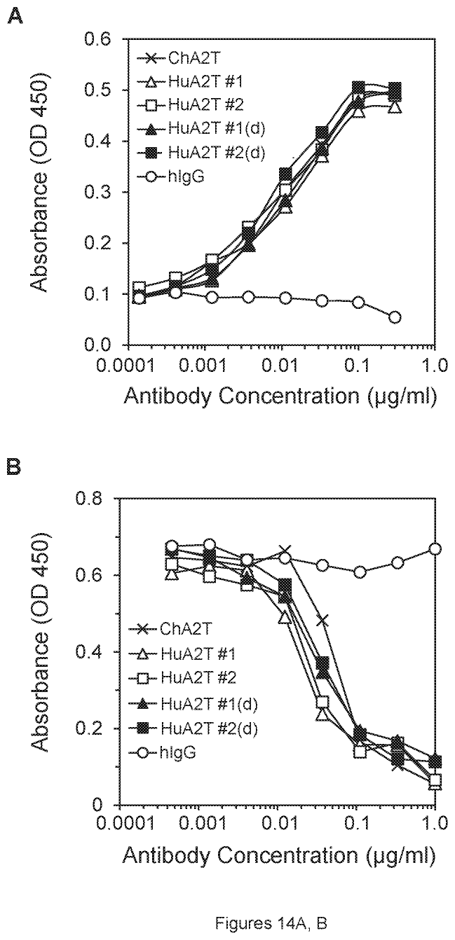

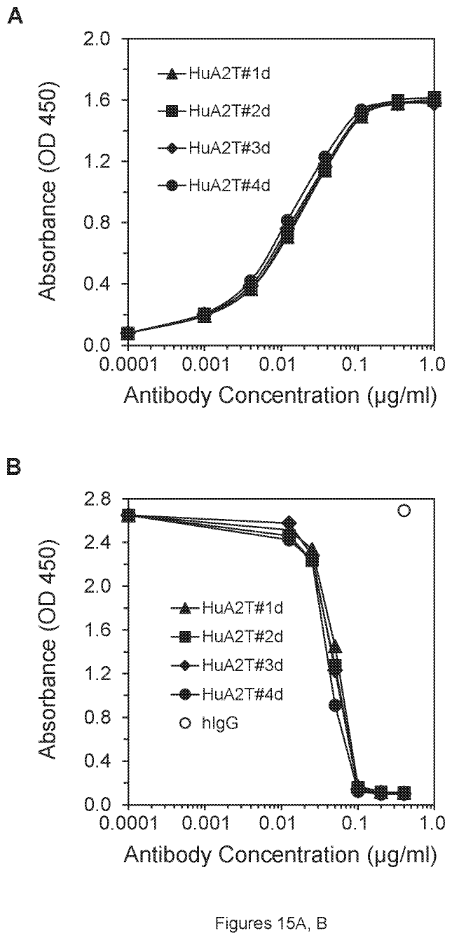

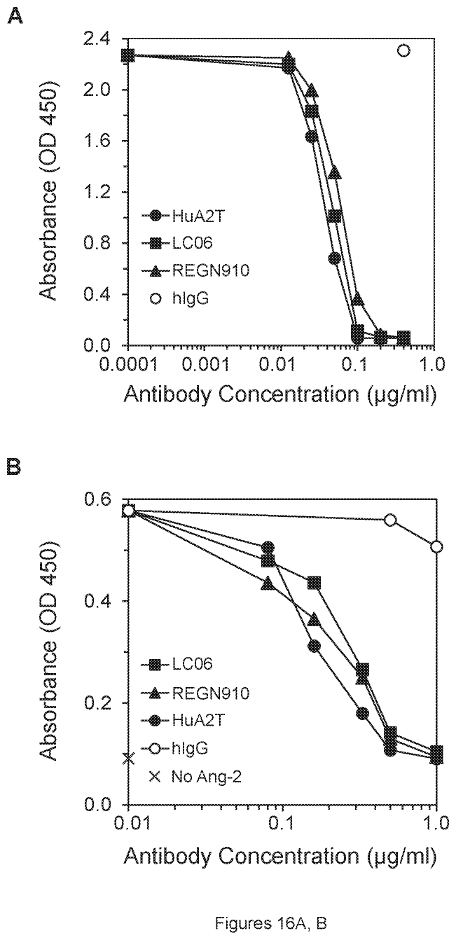

The HuA2T versions with T at position 60 were compared with the respective versions with A at position 60 in binding and blocking assays, and no significant differences were observed, as seen for example in FIG. 14A for binding and FIG. 14B for blocking. Moreover, the HuA2T versions bound Ang-2 as well as ChA2T did (FIG. 14A) and actually blocked binding of Ang-2 to Tie-2 slightly better than ChA2T in the assay (FIG. 14B), indicating no activity was lost during humanization. Since it is preferable not to have glycosylation in an antibody V region due to possible protein heterogeneity and other issues, further studies were conducted with the deglycosylated (d) versions of HuA2T. All four mAbs HuA2T #1(d), #2(d), #3(d) and #4(d) bound (FIG. 15A) and blocked (FIG. 15B) very similarly, with HuA2T #4(d) perhaps just slightly superior to the others. Thus, in all that follows, HuA2T #4(d) will be designated simply as HuA2T. We also showed that the activity of HuA2T in blocking binding of Ang-2 to Tie-2 is similar to the previously described human anti-Ang-2 mAbs REGN910 and LCO6 (FIG. 16A).

Finally, we compared the activity of HuA2T, REGN910 and LCO6 in a more biological assay: inhibition of Ang-2 induced Tie-2 phosphorylation. HEK293 human embryonic kidney cells (ATCC CRL 1573) were first transfected with the Tie-2 gene in an expression vector, so these HEK293-Tie-2 cells expressed full-length human Tie-2 receptor. The cells were grown in DMEM media with 10% fetal calf serum in 24-well plates. The media was replaced with DMEM-0.1% BSA without serum and the cells incubated for 18 hours. Cells were stimulated for 20 min (37° C., 5% CO2) with human recombinant Ang-2 (R&D Systems; 1 μg/mL) in the presence of various concentrations of mAbs. The level of phosphorylated Tie-2 was determined by an ELISA kit following the manufacturer's instructions (R&D Systems #DYC2720). The three mAbs inhibited phosphorylation similarly, with HuA2T slightly better than LCO6 (FIG. 16B).

### Example 10: Bispecific HuVE1/HuA2T Antibody

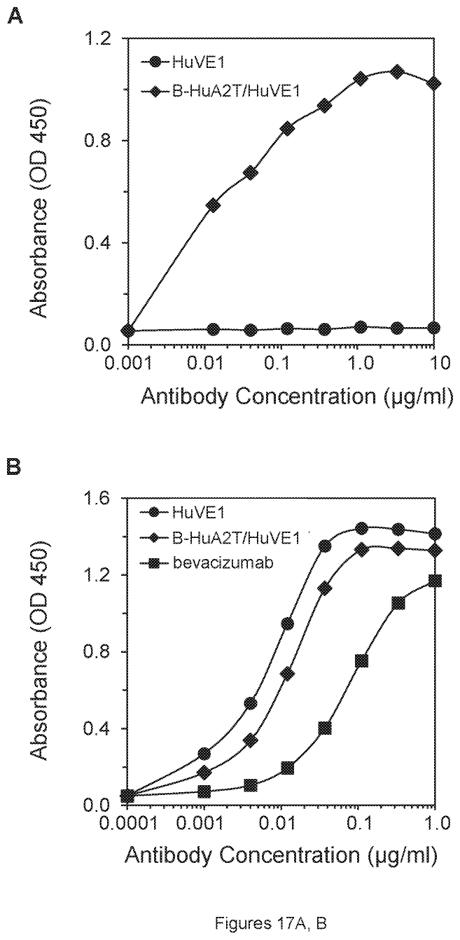

A bispecific antibody designated B-HuA2T/HuVE1 was constructed comprising binding domains from the HuVE1 anti-VEGF mAb and the HuA2T anti-Ang-2 mAb, using the Bs(scFv)4-IgG format illustrated schematically in FIG. 1. With respect to the labeling in FIG. 1A, V_{L}1 and V_{H}1 are respectively HuVE1-L1 and HuVE1-H2 (FIG. 3), while V_{L}2 and V_{H}2 are respectively HuA2T-L1 and HuA2T-H2 (FIG. 13), the linkers between the respective heavy and light chain domains are (G₄S)₃GS, and the constant regions are of human IgG1, kappa isotype. To show that the bispecific B-HuA2T/HuVE1 mAb is able to simultaneously bind VEGF and Ang-2, an ELISA plate was coated with a GST-VEGF (a fusion protein of glutamine synthetase and VEGF), then incubated with increasing concentrations of the bispecific mAb or control mAb HuVE1, followed by hAng-2(F)-KF and detection with HRP-anti-Flag M2 and TMB substrate. Only molecules that can bind both to GST-VEGF on the plate and Ang-2 in solution will give a positive signal in this assay. Such was the case with B-HuA2T/HuVE1 but not with HuVE1 that can only bind VEGF (FIG. 17A).

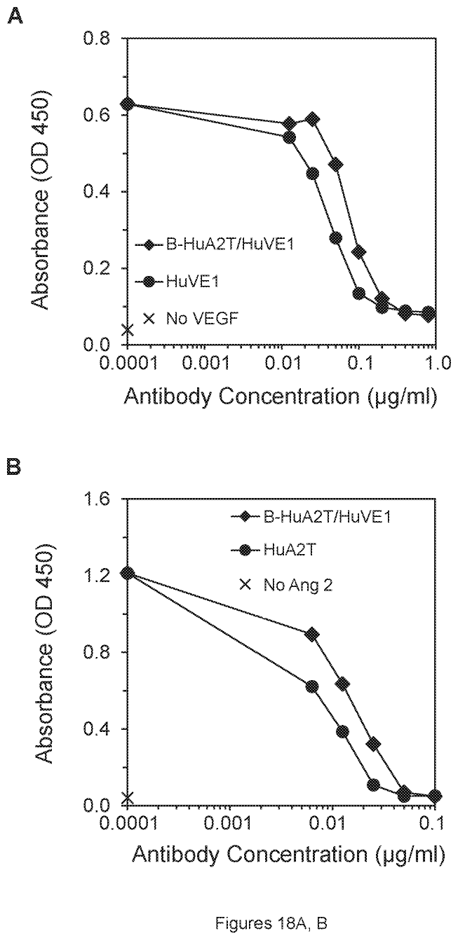

To compare the activity of HuVE1 and HuA2T as individual mAbs with their activity as part of B-HuA2T/HuVE1, we first compared the binding activity of HuVE1 and B-HuA2T/HuVE1 using the VEGF capture assay described in Example 2. The binding activity of B-HuA2T/HuVE1 was reduced only about 2-fold from that of HuVE1 as measured by EC50 and, importantly, was still considerably better than bevacizumab (FIG. 17B), which of course is known to be efficacious against cancer in humans. Similarly, using the binding assay described in Example 8, the binding activity of B-HuA2T/HuVE1 for Ang-2 was reduced about 3-fold from that of HuA2T. Finally, using the blocking assays described in Example 8, the ability of B-HuA2T/HuVE1 to inhibit binding of VEGF to VEGFR2 (FIG. 18A) and to inhibit binding of Ang-2 to Tie-2 (FIG. 18B) was measured: B-HuA2T/HuVE1 was able to essentially completely block binding of both VEGF and Ang-2 to their receptors, although with about 2-fold lower activity than HuVE1 and HuA2T respectively. Since the binding domains of HuVE1 and HuA2T are in single-chain form in B-HuA2T/HuVE1, it is not unexpected that there is some loss of activity.

### Example 11: Ability of VE1 and HuVE1 to Inhibit Growth of Tumor Xenografts

Xenograft experiments are carried out as described previously (Kim et al., Nature 362:841, 1993), with various dosing regimens. Human tumor cells typically grown in complete DMEM medium are harvested in HBSS. Female athymic nude mice (5-6 wks old) are injected subcutaneously with 2-10×10⁶ cells in 0.1 ml of HBSS in the dorsal areas. When the tumor size typically reaches 100 mm³, the mice are grouped randomly and 5 mg/kg (100 μg total) of mAbs are administered i.p. twice per week in a volume of 0.1 ml, or using other dosage regimens as indicated. Tumor sizes are determined twice a week by measuring in two dimensions [length (a) and width (b)]. Tumor volume is calculated according to V=ab²/2 and expressed as mean tumor volume±SEM. The number of mice in each treatment group is typically 5-7 mice. Statistical analysis can be performed, e.g., using Student's t test on the final data point.

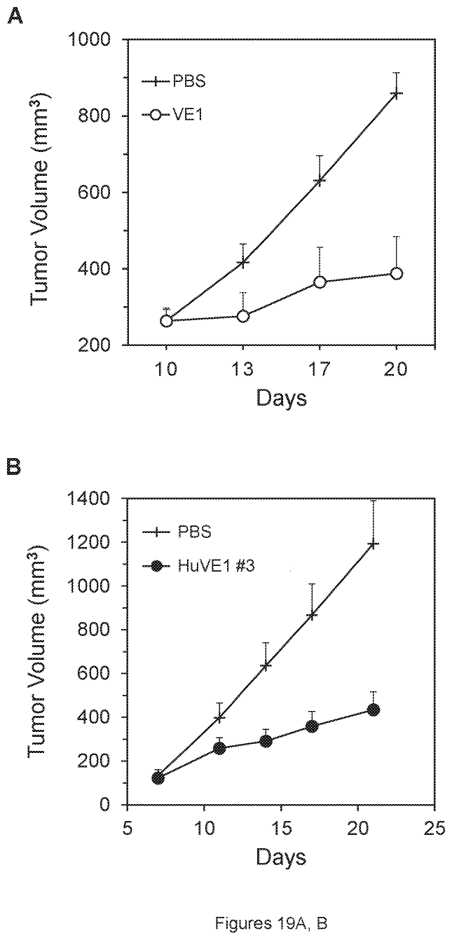

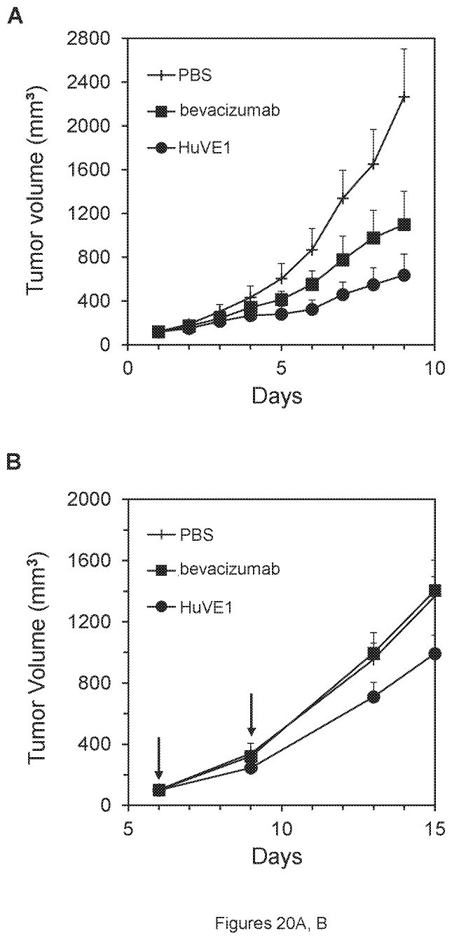

FIG. 19A shows that treatment with VE1 (5 mg/kg, twice per week) inhibited the growth COLO 205 colon tumor (ATCC CCL-222) xenografts. Similarly, FIG. 19B shows that treatment with HuVE1 #3 in the same dosage regimen inhibited the growth of COLO 205 xenografts about as well as VE1. To show that HuVE1 is superior to bevacizumab at inhibition of tumor xenografts in some models, lower doses of the two mAbs were used, since at higher doses, bevacizumab is itself highly effective. In a primary liver tumor model, where the human tumor is passaged in mice and is not converted to a cell line, there was a trend to greater efficacy of HuVE1 relative to bevacizumab (FIG. 20A, p=0.1) when the mAbs were dosed at 2.5 mg/kg twice per week. And whereas bevacizumab was not effective against xenografts of RPMI 4788 colon tumor cells (Roswell Park Institute, referenced in M. Aonuma et al., Anticancer Res 19:4039-4044, 1999) when given at 1 mg/kg on days 6 and 9, HuVE1 was partly effective (FIG. 20B, p=0.01 for HuVE1 vs bevacizumab).

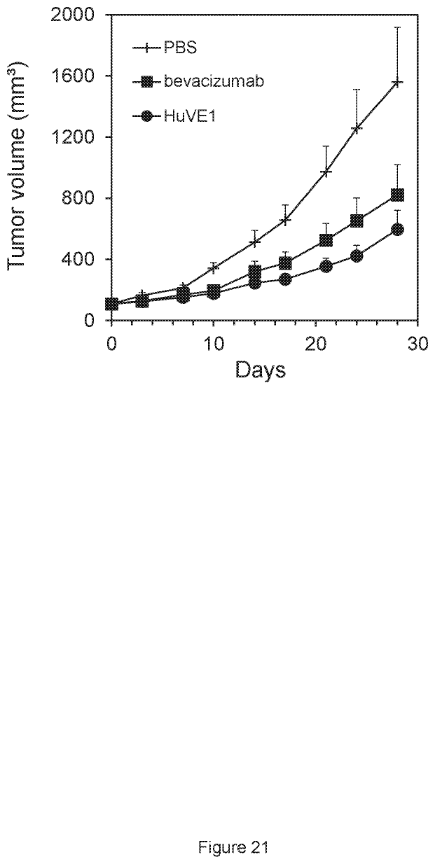

Similarly, in a primary breast tumor xenograft model, there was a trend to greater efficacy of HuVE1 relative to bevacizumab (FIG. 21) when the mAbs were dosed at 5 mg/kg once per week.

Although the invention has been described with reference to the presently preferred embodiments, it should be understood that various modifications can be made without departing from the invention. Unless otherwise apparent from the context any step, element, embodiment, feature or aspect of the invention can be used with any other. All publications, patents and patent applications including accession numbers and the like cited are herein incorporated by reference in their entirety for all purposes to the same extent as if each individual publication, patent and patent application was specifically and individually indicated to be incorporated by reference in its entirety for all purposes. The word “herein” shall indicate anywhere in this patent application, not merely within the section where the word “herein” occurs. If more than one sequence is associated with an accession number at different times, the sequence associated with the accession number as of the effective filing date of this application is intended, the effective filing date meaning the actual filing date or earlier date of a filing of a priority application disclosing the accession number in question.

We claim:

1. A monoclonal antibody (mAb) that binds and neutralizes VEGF and has the same epitope as the VE1 antibody.

2. The mAb of claim 1 comprising a light chain variable region having three CDRs from the light chain variable region sequence of VE1 in FIG. 3A and a heavy chain variable region having three CDRs from the heavy chain variable region sequence of VE1 in FIG. 3B.

3. The mAb of claim 2 which is a humanized antibody.

4. The mAb of claim 2 comprising a light chain variable region with the sequence of HuVE1-L1 or HuVE1-L2 in FIG. 3A and a heavy chain variable region with the sequence of HuVE1-H1 or HuVE1-H2 in FIG. 3B.

5. The mAb of claim 2 which is a Fv, Fab or F(ab′)₂ fragment or single-chain antibody.

6. The mAb of claim 2 which inhibits growth of a human tumor xenograft in a mouse.

7. The mAb of claim 2 which is a bispecific antibody.

8. The mAb of claim 7 which comprises a first binding domain that binds to VEGF and a second binding domain that binds to HGF or FGF2 or Ang-2.

9. The mAb of claim 7 which is a homodimer of monomers, each of which comprises a first binding domain that binds to VEGF and a second binding domain that binds to HGF or FGF2 or Ang-2.

10. A pharmaceutical composition comprising a mAb of claim 2.

11. A method of treating a patient having a disease comprising administering to the patient the pharmaceutical composition of claim 10.

12. The method of claim 11, wherein the disease is cancer.

13. A monoclonal antibody (mAb) that binds and neutralizes Ang-2 and has the same epitope as the A2T antibody.

14. The mAb of claim 13 comprising a light chain variable region having three CDRs from the light chain variable region sequence of A2T in FIG. 13A and a heavy chain variable region having three CDRs from the heavy chain variable region sequence of A2T in FIG. 13B.

15. The mAb of claim 14 which is a humanized antibody.

16. The mAb of claim 15 comprising a light chain variable region with the sequence of HuA2T-L1 or HuA2T-L2 in FIG. 13A and a heavy chain variable region with the sequence of HuA2T-H1 or HuA2T-H2 in FIG. 13B.

17. The mAb of claim 14 which is a bispecific antibody.

18. A pharmaceutical composition comprising a mAb of claim 14.

19. A method of treating a patient having a disease comprising administering the pharmaceutical composition of claim 18.

20. The method of claim 19, wherein the disease is cancer.
